(19) United States
(12) Patent Application Publication
(10) Pub. No.: US 2018/0142296 A1
(43) Pub. Date: May 24, 2018

(54) MULTIPLEXED OPTIMIZED MISMATCH AMPLIFICATION (MOMA)-REAL TIME PCR FOR ASSESSING CELL-FREE DNA

(71) Applicant: Medical College of Wisconsin, Inc., Milwaukee, WI (US)

(72) Inventors: Aoy Tomita MITCHELL, Elm Grove, WI (US); Michael MITCHELL, Elm Grove, WI (US); Karl STAMM, Wauwatosa, WI (US)

(73) Assignee: Medical College of Wisconsin, Inc., Miwaukee, WI (US)

(21) Appl. No.: 15/570,560

(22) PCT Filed: Apr. 29, 2016
(86) PCT No.: PCT/US2016/030313

§ 371 Date: Oct. 30, 2017

Related U.S. Application Data
(60) Provisional application No. 62/155,453, filed on Apr. 30, 2015.

(51) Int. Cl. C12Q 1/6881; C12Q 1/6851; C12Q 1/6858

(52) U.S. Cl. CPC C12Q 1/6881; C12Q 1/6851; C12Q 2600/156; C12Q 2600/16; C12Q 2600/118; C12Q 1/6858

(57) ABSTRACT

This invention relates to methods and compositions for assessing an amount of non-native nucleic acids in a sample, such as from a subject. The methods and compositions provided herein can be used to determine risk of a condition, such as transplant rejection, in subject.

### RELATED APPLICATIONS

This application claims the benefit under 35 U.S.C. 119(e) of the filing date of U.S. Provisional Application 62/155,453, filed Apr. 30, 2015, the contents of which are incorporated herein by reference in their entirety.

### FIELD OF THE INVENTION

This invention relates to methods and compositions for assessing an amount of non-native nucleic acids in a sample from a subject. The methods and compositions provided herein can be used to determine risk of a condition, such as transplant rejection. This invention further relates to methods and compositions for assessing the amount of non-native cell-free deoxyribonucleic acid (non-native cell-free DNA, such as donor-specific cell-free DNA) using multiplexed optimized mismatch amplification (MOMA).

### BACKGROUND OF THE INVENTION

The ability to detect and quantify non-native nucleic acids in a sample may permit the early detection of a condition, such as transplant rejection. Current methods for quantitative analysis of heterogeneous nucleic acid populations (e.g., a mixture of native and non-native nucleic acids), however, are limited.

### SUMMARY OF INVENTION

The present disclosure is based, at least in part on the surprising discovery that multiplexed optimized mismatch amplification can be used to quantify low frequency non-native nucleic acids in samples from a subject. Multiplexed optimized mismatch amplification embraces the design of primers that can include a 3′ penultimate mismatch for the amplification of a specific sequence but a double mismatch relative to an alternate sequence. Amplification with such primers can permit the quantitative determination of amounts of non-native nucleic acids in a sample, even where the amount of non-native nucleic acids are, for example, below 1%, or even 0.5%, in a heterogeneous population of nucleic acids.

Provided herein are methods, compositions and kits related to such optimized amplification. The methods, compositions or kits can be any one of the methods, compositions or kits, respectively, provided herein, including any one of those of the examples and drawings.

In one aspect, a method of assessing an amount of non-native nucleic acids in a sample from a subject is provided. In one embodiment the method comprises, for each of a plurality of single nucleotide variant (SNV) targets, obtaining results from an amplification-based quantification assay, such as a polymerase chain reaction (PCR) quantification assay, on a sample, or portion thereof, with at least one primer pair, wherein the at least one primer pair comprises a forward primer and a reverse primer, wherein the at least one primer pair comprises a primer with a 3′ mismatch (e.g., penultimate mismatch) relative to one sequence (e.g., allele) of the SNV target but a 3′ double mismatch relative to another sequence (e.g., allele) of the SNV target and specifically amplifies the one sequence (e.g., allele) of the SNV target.

In one embodiment of any one of the methods provided herein, the method further comprises, for each SNV target, obtaining results from a quantification assay with at least one another primer pair, wherein the at least one another primer pair comprises a forward primer and a reverse primer, wherein the at least one another primer pair specifically amplifies another sequence (e.g., allele) of the SNV target.

In one embodiment, a method of assessing an amount of non-native nucleic acids in a sample from a subject, for each of a plurality of single nucleotide variant (SNV) targets, performing an amplification-based quantification assay, such as a PCR quantification assay, on the sample, or portion thereof, with at least two primer pairs, wherein each primer pair comprises a forward primer and a reverse primer, wherein one of the at least two primer pairs comprises a 3′ mismatch (e.g., penultimate) relative to one sequence (e.g., allele) of the SNV target but a 3′ double mismatch relative to another sequence (e.g., allele) of the SNV target and specifically amplifies the one sequence (e.g., allele) of the SNV target, and another of the at least two primer pairs specifically amplifies the another sequence (e.g., allele) of the SNV target is provided.

In one embodiment, a method of assessing an amount of non-native nucleic acids in a sample from a subject, comprising obtaining results from an amplification-based amplification assay, such as a polymerase chain reaction (PCR) quantification assay, for each of a plurality of single nucleotide variant (SNV) targets, performed on the sample, or portion thereof, with at least two primer pairs, wherein each primer pair comprises a forward primer and a reverse primer, wherein one of the at least two primer pairs comprises a 3′ mismatch (e.g., penultimate) relative to one sequence (e.g., allele) of the SNV target but a 3′ double mismatch relative to another sequence (e.g., allele) of the SNV target and specifically amplifies the one sequence (e.g., allele) of the SNV target, and another of the at least two primer pairs specifically amplifies the another sequence (e.g., allele) of the SNV target is provided.

In one embodiment, a method of assessing the amount of non-native nucleic acids in a sample, such as from a subject, the sample comprising non-native and native nucleic acids, the method comprising for a plurality of SNV targets, for each such SNV target, obtaining results from an amplification-based quantification assay, such as a polymerase chain reaction (PCR) assay on the sample with at least one primer pair as provided herein, such as at least two primer pairs, wherein each primer pair comprises a forward primer and a reverse primer, selecting informative results based on the genotype of the native nucleic acids and/or non-native nucleic acids, and determining the amount of the non-native nucleic acids in the sample based on the informative results is provided. In one embodiment, the method further comprises identifying the plurality of SNV targets. In one embodiment, the method further comprises inferring the genotype of the non-native nucleic acids. In one embodiment, the method further comprises providing the results.

In one embodiment, a method of assessing an amount of non-native nucleic acids in a sample from a subject, the method comprising obtaining results from 1) an amplification-based quantification assay, such as a PCR quantification assay, for each of a plurality of SNV targets, performed on a sample, or portion thereof, with at least one primer pair, such as at least two primer pairs, wherein each primer pair comprises a forward primer and a reverse primer, wherein one of the at least one, such as at least two, primer pair, comprises a 3′ mismatch (e.g., penultimate) relative to one sequence (e.g., allele) of the SNV target but a 3′ double mismatch relative to another sequence (e.g., allele) of the SNV target and specifically amplifies the one sequence (e.g., allele) of the SNV target and 2) a determination of informative results based on the native genotype and/or a prediction of the likely non-native genotype is provided. In one embodiment, when there are at least two primer pairs, the another primer pair specifically amplifies the another sequence (e.g., allele) of each SNV target and quantification results are obtained with the another primer pair for each of the SNV targets.

In one embodiment, a method of assessing an amount of non-native nucleic acids in a sample from a subject the method comprising obtaining results from 1) an amplification-based quantification assay, such as a PCR quantification assay, for each of a plurality of SNV targets, performed on a sample, or portion thereof, with at least two primer pairs, wherein each primer pair comprises a forward primer and a reverse primer, wherein one of the at least two primer pairs comprises a 3′ mismatch (e.g., penultimate) relative to one sequence (e.g., allele) of the SNV target but a 3′ double mismatch relative to another sequence (e.g., allele) of the SNV target and specifically amplifies the one sequence (e.g., allele) of the SNV target, and another of the at least two primer pairs specifically amplifies the another sequence (e.g., allele) of the SNV target, and 2) a determination of informative results based on the native genotype and/or a prediction of the likely non-native genotype.

In one embodiment of any one of the methods, compositions or kits provided herein, further comprising at least one another primer pair for each SNV target and/or obtaining results with an amplification-based quantification assay, such as a PCR quantification assay therewith. In one embodiment of any one of the methods, compositions or kits provided herein, the at least one another primer pair comprises a 3′ mismatch (e.g., penultimate) relative to another sequence (e.g., allele) of the SNV target but a 3′ double mismatch relative to the one sequence (e.g., allele) of the SNV target and specifically amplifies the another sequence (e.g., allele) of the SNV target.

In one embodiment of any one of the methods provided, the method further comprises assessing the amount of non-native nucleic acids based on the results. In one embodiment of any one of the methods provided, the results are informative results.

In one embodiment of any one of the methods provided, the method further comprises selecting informative results of the amplification-based quantification assays, such as PCR quantification assays. In one embodiment of any one of the methods provided, the selected informative results are averaged.

In one embodiment of any one of the methods provided, the informative results of the amplification-based quantification assays, such as PCR quantification assays are selected based on the genotype of the non-native nucleic acids and/or native nucleic acids.

In one embodiment of any one of the methods provided, the method further comprises obtaining the genotype of the non-native nucleic acids and/or native nucleic acids.

In one embodiment of any one of the methods provided, the method further comprises selecting informative results based on the native genotype and/or prediction of the likely non-native genotype. In one embodiment of any one of the methods provided, when the genotype of the non-native nucleic acids is not known or obtained, the method further comprises assessing results based on a prediction of the likely non-native genotype. In one embodiment of any one of the methods provided, the method comprises the amount of the non-native nucleic acids in the sample based on the informative results and prediction. In one embodiment of any one of the methods provided, the assessing or prediction is performed with an expectation-maximization algorithm. In one embodiment of any one of the methods provided, expectation-maximization is used to predict the likely non-native genotype.

In one embodiment of any one of the methods provided, maximum likelihood is used to calculate the amount of non-native nucleic acids.

In one embodiment of any one of the methods provided, the method further comprises obtaining the plurality of SNV targets.

In one embodiment of any one of the methods provided, the method further comprises obtaining the at least one, such as at least two primer pairs, for each of the plurality of SNV targets.

In one embodiment of any one of the methods provided, the method further comprises obtaining or providing the results. In one embodiment of any one of the methods provided, the results are informative results. In one embodiment of any one of the methods provided, the results comprise the amount of the non-native nucleic acids in the sample.

In one embodiment of any one of the methods provided herein, the results are provided in a report. In one aspect, such a report is provided herein. In one embodiment of any one of the methods or reports provided, the results are informative results. In one embodiment of any one of the methods or report provided, the results comprise the amount of the non-native nucleic acids in the sample.

In one embodiment of any one of the methods provided herein, the results are obtained from a report. In one embodiment of any one of the reports provided, the report is given in electronic form. In one embodiment of any one of the reports provided, the report is a hard copy. In one embodiment of any one of the reports provided, the report is given orally.

In one embodiment of any one of the methods provided herein, the results are or can be used to determine the amount of non-native nucleic acids in the sample. In one embodiment of any one of the methods provided, the results are informative results.

In one embodiment of any one of the methods provided herein, the method further comprises determining the amount of the non-native nucleic acids in the sample, such as based on the results. In one embodiment of any one of the methods provided, the results are informative results. In one embodiment of any one of the methods provided herein, the amount of the non-native nucleic acids in the sample is based on the results of the amplification-based quantification assays, such as PCR quantification assays. In one embodiment of any one of the methods provided herein, the results are informative results of the amplification-based quantification assays, such as PCR quantification assays.

In one embodiment of any one of the method, compositions, kits or reports provided herein, the amount is the ratio or percentage of non-native nucleic acids to native nucleic acids.

In one embodiment of any one of the methods, compositions or kits provided, there is at least one primer pair, at least two primer pairs, at least three primer pairs, at least four primer pairs or more per SNV target. In one embodiment of any one of the methods, compositions or kits provided, the plurality of SNV targets is at least 45, 48, 50, 55, 60, 65, 70, 75, 80, 85 or 90 or more. In one embodiment of any one of the methods, compositions or provided, the plurality of SNV targets is at least 90, 95 or more targets. In one embodiment of any one of the methods, compositions or kits provided, the plurality of SNV targets is less than 105 or 100 targets.

In one embodiment of any one of the methods, compositions or kits provided, the mismatched primer(s) is/are the forward primer(s). In one embodiment of any one of the methods, compositions or kits provided, the reverse primers for the primer pairs for each SNV target is the same.

In one embodiment of any one of the methods provided, the amount of non-native nucleic acids in the sample is at least 0.005%. In one embodiment of any one of the methods provided, the amount of non-native nucleic acids in the sample is at least 0.01%. In one embodiment of any one of the methods provided, the amount of non-native nucleic acids in the sample is at least 0.03%. In one embodiment of any one of the methods provided, the amount of non-native nucleic acids in the sample is at least 0.05%. In one embodiment of any one of the methods provided, the amount of non-native nucleic acids in the sample is at least 0.1%. In one embodiment of any one of the methods provided, the amount of non-native nucleic acids in the sample is at least 0.3%. In one embodiment of any one of the methods provided, the amount of non-native nucleic acids in the sample is less than 1.5%. In one embodiment of any one of the methods provided, the amount of non-native nucleic acids in the sample is less than 1.3%. In one embodiment of any one of the methods provided, the amount of non-native nucleic acids in the sample is less than 1%. In one embodiment of any one of the methods provided, the amount of non-native nucleic acids in the sample is less than 0.5%.

In one embodiment of any one of the methods provided, the sample comprises cell-free DNA sample and the amount is an amount of non-native cell-free DNA.

In one embodiment of any one of the methods provided, the subject is a transplant recipient, and the amount of non-native nucleic acids is an amount of donor-specific cell-free DNA.

In one embodiment of any one of the methods provided, the transplant recipient is a heart transplant recipient. In one embodiment of any one of the methods provided, the transplant recipient is a pediatric transplant recipient.

In one embodiment of any one of the methods provided, the plurality of amplification-based quantification assays, such as PCR quantification assays, are real time PCR assays or digital PCR assays.

In one embodiment of any one of the methods provided, the method further comprises determining a risk in the subject based on the amount of non-native nucleic acids in the sample. In one embodiment of any one of the methods provided, the risk is a risk associated with a transplant. In one embodiment of any one of the methods provided, the risk associated with a transplant is risk of transplant rejection, an anatomical problem with the transplant or injury to the transplant. In one embodiment of any one of the methods provided herein, the injury to the transplant is initial or ongoing injury. In one embodiment of any one of the methods provided herein, the risk associated with the transplant is indicative of the severity of the injury.

In one embodiment of any one of the methods provided, the risk is increased if the amount of non-native nucleic acids is greater than a threshold value. In one embodiment of any one of the methods provided, the risk is decreased if the amount of non-native nucleic acids is less than a threshold value.

In one embodiment of any one of the methods provided, where the risk is the risk associated with the heart transplant rejection, the threshold value is 1%. In one embodiment of any one of the methods provided, where the risk is the risk associated with the heart transplant rejection, the threshold value is 1.3%.

In one embodiment of any one of the methods provided, the method further comprises selecting a treatment for the subject based on the amount of non-native nucleic acids.

In one embodiment of any one of the methods provided, the method further comprises treating the subject based on the amount of non-native nucleic acids.

In one embodiment of any one of the methods provided, the method further comprises providing information about a treatment to the subject based on the amount of non-native nucleic acids.

In one embodiment of any one of the methods provided, method further comprises monitoring or suggesting the monitoring of the amount of non-native nucleic acids in the subject over time.

In one embodiment of any one of the methods provided, the method further comprises assessing the amount of non-native nucleic acids in the subject at a subsequent point in time.

In one embodiment of any one of the methods provided, the method further comprises obtaining another sample from the subject, such as at a subsequent point in time, and performing a test on the sample, such as any one of the methods provided herein.

In one embodiment of any one of the methods provided, the method further comprises evaluating an effect of a treatment administered to the subject based on the amount of non-native nucleic acids.

In one embodiment of any one of the methods provided, the treatment is an anti-rejection therapy.

In one embodiment of any one of the methods provided, the method further comprises providing or obtaining the sample or a portion thereof.

In one embodiment of any one of the methods provided, the method further comprises extracting nucleic acids from the sample.

In one embodiment of any one of the methods provided, the method further comprises an amplification step. In one embodiment of any one of the methods provided, the amplification is performed prior to the quantification assay(s).

In one embodiment of any one of the methods provided, the sample comprises blood, plasma, serum or urine.

In one embodiment of any one of the methods provided, the sample is obtained or is one that was obtained from the subject within 10 days of a heart transplant.

In one aspect, a method of determining a plurality of SNV targets, comprising a) identifying a plurality of highly heterozygous SNVs in a population of individuals, b) designing one or more primers spanning each SNV, c) selecting sufficiently specific primers, d) evaluating multiplexing capabilities of primers, such as at a common melting temperature and/or in a common solution, and e) identifying sequences that are evenly amplified with the primers or a subset thereof. In one embodiment of any one of the methods provided herein, the method further comprises computing the melting temperatures and/or GC % of the selected primers. In one embodiment of any one of the methods provided herein, the method further comprises filtering for moderate range sequences.

In one embodiment of any one of the methods provided herein, step a) further comprises selecting SNVs with a Hardy-Weinberg p>0.25 and/or excluding those associated with difficult regions. In one embodiment of any one of the methods provided herein, the difficult regions are syndromic regions and/or low complexity regions.

In one embodiment of any one of the methods provided, the one or more primers of step b) span a 70 bp window and/or the one or more primers are 16-26 bps in length, such as 20-26 bps in length.

In one embodiment of any one of the methods provided, the sufficiently specific primers of step c) are identified with a BLAST analysis. In one embodiment of any one of the methods provided, the BLAST analysis is against GCRh37.

In one embodiment of any one of the methods provided, the method includes or further includes performing an iterated genetic algorithm and/or simulated annealing.

In one embodiment of any one of the methods provided, the method further comprises obtaining at least one primer pair for each identified SNV target wherein the at least one primer pair comprises a 3′ mismatch (e.g., penultimate) relative to one sequence (e.g., allele) of the SNV target but a 3′ double mismatch relative to another sequence (e.g., allele) of the SNV target and specifically amplifies the one sequence (e.g., allele) of the SNV target.

In one embodiment of any one of the methods provided, the method further comprises obtaining another primer pair for each identified SNV target, wherein the another primer pair specifically amplifies the another sequence (e.g, allele) of the SNV target.

In one embodiment of any one of the methods provided, wherein the another primer pair comprises a 3′ mismatch (e.g., penultimate) relative to the another sequence (e.g., allele) of the SNV target but a 3′ double mismatch relative to the one sequence (e.g., allele) of the SNV target.

In one embodiment of any one of the methods provided, wherein there are at least 45, 48, 50, 55, 60, 65, 70, 75, 80, 85 or 90 or more SNV targets. In one embodiment of any one of the methods provided, there are at least 90, 95 or more SNV targets. In one embodiment of any one of the methods provided, there are less than 105 or 100 SNV targets.

In one embodiment of any one of the methods provided, the method further comprises providing the at least one primer pair for each SNV target.

In one aspect, a method of inferring non-native nucleic acid genotypes comprising, obtaining non-native nucleic acid levels, such as informative non-native nucleic acid levels, for each of a plurality of SNV targets and assigning each level to one of at least two distributions, one of which is for fully informative levels and the other is for half informative levels, such as with a maximization step. In one embodiment of any one of the methods provided, when the informativity of the levels obtained is not yet known, the at least two distributions are three distributions, one of which is for fully informative levels, another is for half informative levels and the last is for non-informative or background levels. In one embodiment, where informative non-native nucleic acid levels are obtained for each of a plurality of SNV targets, each level is assigned as either fully informative or half informative, such as with a maximization step.

In one embodiment of any one of the methods provided, the informative non-native nucleic acid levels are obtained by removing levels that are determined to be of native nucleic acids and/or that represent a no call or erroneous call.

In one embodiment of any one of the methods provided, the method further comprises removing levels that represent a no call or erroneous call.

In one embodiment of any one of the methods provided, the levels are obtained with sequencing, such as next generation sequencing, such as on a sample from a subject.

In one embodiment of any one of the methods provided the levels are obtained from an amplification-based quantification assay, such as a PCR quantification assay. In one embodiment of any one of the methods provided, the levels are obtained from any one of the methods provided. In one embodiment of any one of the methods, the levels are obtained by performing the amplification-based quantification assay, such as a PCR quantification assay, for each of the plurality of SNV targets. In one embodiment of any one of the methods provided, the levels are obtained by performing any one of the methods provided herein. In one embodiment of any one of the methods provided, the levels are obtained by performing a quantification assay, such as a PCT quantification assay, using any one of the compositions of primers provided herein.

In one embodiment of any one of the methods provided, the amplification-based quantification assay, such as PCR quantification assay is performed with at least two primer pairs for each of the plurality of SNV targets, wherein each primer pair comprises a forward primer and a reverse primer, wherein one of the at least two primer pairs comprises a 3′ (e.g., penultimate) mismatch relative to one sequence (e.g., allele) of the SNV target but a 3′ double mismatch relative to another sequence (e.g., allele) of the SNV target and specifically amplifies the one sequence (e.g., allele) of the SNV target, and another of the at least two primer pairs specifically amplifies the another sequence (e.g., allele) of the SNV target.

In one embodiment of any one of the methods provided, the another primer pair of the at least two primer pairs also comprises a 3′ (e.g., penultimate) mismatch relative to the another sequence (e.g., allele) of the SNV target but a 3′ double mismatch relative to the one sequence (e.g., allele) of the SNV target and specifically amplifies the another sequence (e.g., allele) of the SNV target.

In one embodiment of any one of the methods provided, the method further comprises providing the assigned levels. In one embodiment of any one of the methods provided, the assigned levels are provided in a report.

In one embodiment of any one of the methods provided, the method further comprises obtaining the amount of non-native nucleic acids based on the assignment of the levels.

In one embodiment of any one of the methods provided, the method further comprises providing the amount of non-native nucleic acids based on the assignment of the levels. In one embodiment of any one of the methods provided, the amount of non-native nucleic acids based on the assignment of the levels is provided in a report.

In one aspect, a method of obtaining any one of the sets of assigned levels or combination thereof or amount of non-nucleic acids based on the assignment according to any one of the methods provided herein, and assessing a risk in a subject based on the levels or amount is provided.

In one embodiment of any one of the methods provided, a treatment or information about a treatment is given to the subject based on the assessed risk. In one embodiment of any one of the methods provided, the treatment is an anti-rejection therapy.

In one embodiment of any one of the methods provided, the method further comprises monitoring or suggesting the monitoring of the amount of non-native nucleic acids in the subject over time. In one embodiment of any one of the methods provided, the method further comprises determining the amount of non-native nucleic acids in the subject at a subsequent point in time. In one embodiment of any one of the methods provided, the amount is determined with any one of the methods provided herein.

In one aspect a composition or kit comprising at least one primer pair, for each of a plurality of SNV targets, wherein each primer pair comprises a 3′ mismatch (e.g., penultimate) relative to one sequence (e.g., allele) of a SNV target but a 3′ double mismatch relative to another sequence (e.g., allele) of the SNV target and specifically amplifies the one sequence (e.g., allele) of the SNV target is provided. In one embodiment of any one of the methods, compositions or kits provided, further comprising at least one another primer pair for each of the plurality of SNV targets wherein the at least one another primer pair specifically amplifies the another sequence (e.g., allele) of the SNV target. In one embodiment of any one of the methods, compositions or kits provided, the at least one another primer pair comprises a 3′ mismatch (e.g., penultimate) relative to the another sequence (e.g., allele) of a SNV target but a 3′ double mismatch relative to the another sequence (e.g., allele) of the SNV target and specifically amplifies the another sequence (e.g., allele) of the SNV target.

In one embodiment of any one of the methods, compositions or kits provided, each primer pair comprises a 3′ mismatch (e.g., penultimate) relative to one sequence (e.g., allele) of a SNV target but a 3′ double mismatch relative to another sequence (e.g., allele) of the SNV target and specifically amplifies the one allele of the SNV target.

In one embodiment of any one of the methods, compositions or kits provided, there is at least one primer pair, at least two primer pairs, at least three primer pairs, at least four primer pairs or more per SNV target. In one embodiment of any one of the compositions or kits provided herein, there is at least two primer pairs for each SNV target. In one embodiment of any one of the methods, compositions or kits provided, there is at least 45, 48, 50, 55, 60, 65, 70, 75, 80, 85 or 90 or more SNV targets. In one embodiment of any one of the methods, compositions or provided, there is at least 90, 95 or more targets. In one embodiment of any one of the methods, compositions or kits provided, there is less than 105 or 100 SNV targets.

In one embodiment of any one of the compositions or kits provided, the composition or kit comprises a buffer.

In one embodiment of any one of the compositions or kits provided, the composition or kit comprises a polymerase.

In one embodiment of any one of the compositions or kits provided, the composition or kit comprises a probe. In one embodiment of any one of the compositions or kits provided, wherein the probe is a fluorescent probe.

In one embodiment of any one of the compositions or kits provided, the composition or kit comprises instructions for use. In one embodiment of any one of the compositions or kits provided, wherein the instructions for use are instructions for determining the amount of non-native nucleic acids in a sample. In one embodiment of any one of the compositions or kits provided herein, the instructions for use comprises instructions for performing any one of the methods provided herein.

In one embodiment, any one of the embodiments for the methods provided herein can be an embodiment for any one of the compositions, kits or reports provided. In one embodiment, any one of the embodiments for the compositions, kits or reports provided herein can be an embodiment for any one of the methods provided herein.

### BRIEF DESCRIPTION OF DRAWINGS

The accompanying drawings are not intended to be drawn to scale. The figures are illustrative only and are not required for enablement of the disclosure.

FIG. 1 provides an exemplary, non-limiting diagram of MOMA primers. In a polymerase chain reaction (PCR) assay, extension of the sequence containing SNV A is expected to occur, resulting in the detection of SNV A, which may be subsequently quantified. Extension of the SNV B, however, is not expected to occur due to the double mismatch.

FIG. 2 provides exemplary amplification traces.

FIG. 3 shows results from a reconstruction experiment demonstrating proof of concept.

FIG. 4 provides the percent cell-free DNA measured with plasma samples from transplant recipient patients. All data comes from patients who have had biopsies. Dark points denote rejection.

FIG. 5 provides further data from a method as provided herein on plasma samples. After transplant surgery, the donor percent levels drop off.

FIG. 6 demonstrates the use of expectation maximization to predict non-native donor genotype when unknown. Black=background, Green=half informative, Red=fully informative, Dashed line=first iteration, Solid line=second iteration, Final call=10%.

FIG. 7 demonstrates the use of expectation maximization to predict non-native donor genotype when unknown. Black=background, Green=half informative, Red=fully informative, Final call=5%.

FIG. 8 provides reconstruction experiment data demonstrating the ability to predict the non-native donor genotype when unknown. Data have been generated with a set of 95 SNV targets.

FIG. 9 provides the average background noise for 104 MOMA targets.

FIG. 10 provides further examples of the background noise for methods using MOMA.

FIGS. 11-30 illustrate the benefit of having the probe on the same strand as the mismatch primer in some embodiments.

### DETAILED DESCRIPTION OF THE INVENTION

Aspects of the disclosure relate to methods for the sensitive detection and/or quantification of non-native nucleic acids in a sample. Non-native nucleic acids, such as non-native DNA, may be present in individuals in a variety of situations including following organ transplantation. The disclosure provides techniques to detect, analyze and/or quantify non-native nucleic acids, such as non-native cell-free DNA concentrations, in samples obtained from a subject.

As used herein, “non-native nucleic acids” refers to nucleic acids that are from another source or are mutated versions of a nucleic acid found in a subject (with respect to a specific sequence). “Native nucleic acids”, therefore, are nucleic acids that are not from another source and are not mutated versions of a nucleic acid found in a subject (with respect to a specific sequence). In some embodiments, the non-native nucleic acid is non-native cell-free DNA. “Cell-free DNA” (or cf-DNA) is DNA that is present outside of a cell, e.g., in the blood, plasma, serum, urine, etc. of a subject. Without wishing to be bound by any particular theory or mechanism, it is believed that cf-DNA is released from cells, e.g., via apoptosis of the cells. An example of non-native nucleic acids are nucleic acids that are from a donor of a transplant in a transplant recipient subject. As used herein, the compositions and methods provided herein can be used to determine an amount of cell-free DNA from a non-native source, such as DNA specific to a donor or donor-specific cell-free DNA (e.g., donor-specific cfDNA).

Provided herein are methods and compositions that can be used to measure nucleic acids with differences in sequence identity. In some embodiments, the difference in sequence identity is a single nucleotide variant (SNV); however, wherever a SNV is referred to herein any difference in sequence identity between native and non-native nucleic acids is intended to also be applicable. Thus, any one of the methods or compositions provided herein may be applied to native versus non-native nucleic acids where there is a difference in sequence identity. As used herein, “single nucleotide variant” refers to a nucleic acid sequence within which there is sequence variability at a single nucleotide. In some embodiments, the SNV is a biallelic SNV, meaning that there is one major allele and one minor allele for the SNV. In some embodiments, the SNV may have more than two alleles, such as within a population. In some embodiments, the SNV is a mutant version of a sequence, and the non-native nucleic acid refers to the mutant version, while the native nucleic acid refers to the non-mutated version (such as wild-type version). Such SNVs, thus, can be mutations that can occur within a subject and which can be associated with a disease or condition. Generally, a “minor allele” refers to an allele that is less frequent, such as in a population, for a locus, while a “major allele” refers to the more frequent allele, such as in a population. The methods and compositions provided herein can quantify nucleic acids of major and minor alleles within a mixture of nucleic acids even when present at low levels, in some embodiments.

The nucleic acid sequence within which there is sequence identity variability, such as a SNV, is generally referred to as a “target”. As used herein, a “SNV target” refers to a nucleic acid sequence within which there is sequence variability at a single nucleotide, such as in a population of individuals or as a result of a mutation that can occur in a subject and that can be associate with a disease or condition. The SNV target has more than one allele, and in preferred embodiments, the SNV target is biallelic. In some embodiments of any one of the methods provided herein, the SNV target is a SNP target. In some of these embodiments, the SNP target is biallelic. It has been discovered that non-native nucleic acids can be quantified even at extremely low levels by performing amplification-based quantitative assays, such as PCR assays with primers specific for SNV targets. In some embodiments, the amount of non-native nucleic acids is determined by attempting amplification-based quantitative assays, such as quantitative PCR assays, with primers for a plurality of SNV targets. A “plurality of SNV targets” refers to more than one SNV target where for each target there are at least two alleles. Preferably, in some embodiments, each SNV target is expected to be biallelic and a primer pair specific to each allele of the SNV target is used to specifically amplify nucleic acids of each allele, where amplification occurs if the nucleic acid of the specific allele is present in the sample. In some embodiments, the plurality of SNV targets are a plurality of sequences within a subject that can be mutated and that if so mutated can be indicative of a disease or condition in the subject. As used herein, one allele may be the mutated version of a target sequence and another allele is the non-mutated version of the sequence.

In some embodiments, the amplification-based quantitative assay, such as quantitative PCR, is performed with primer pairs for at least 40, 45, 50, 55, 60, 65, 70, 75, 80, 85, 90, 91, 92, 93, 94, 95 or more targets. In some embodiments, the quantitative assay is performed with primer pairs for fewer than 105, 104, 103, 102, 101, 100, 99, 98 or 97 targets. In some embodiments, sufficient informative results are obtained with primer pairs for between 40-105, 45-105, 50-105, 55-105, 60-105, 65-105, 70-105, 75-105, 80-105, 85-105, 90-105, 90-104, 90-103, 90-102, 90-101, 90-100, 90-99, 91-99, 92-99, 93, 99, 94-99, 95-99, or 90-95 targets. In some embodiments, sufficient informative results are obtained with primer pairs for between 40-99, 45-99, 50-99, 55-99, 60-99, 65-99, 70-99, 75-99, 80-99, 85-99, 90-99, 90-99, 90-98, 90-97 or 90-96 targets.

“Informative results” as provided herein are the results that can be used to quantify the level of non-native or native nucleic acids in a sample. Generally, informative results exclude the results where the native nucleic acids are heterozygous for a specific SNV target as well as “no call” or erroneous call results. From the informative results, allele percentages can be calculated using standard curves, in some embodiments of any one of the methods provided. In some embodiments of any one of the methods provided, the amount of non-native and/or native nucleic acids represents an average across informative results for the non-native and/or native nucleic acids, respectively.

The amount, such as ratio or percentage, of non-native nucleic acids may be determined with the quantities of the major and minor alleles as well as the genotype of the native and/or non-native nucleic acids. For example, results where the native nucleic acids are heterozygous for a specific SNV target can be excluded with knowledge of the native genotype. Further, results can also be assessed with knowledge of the non-native genotype. In some embodiments of any one of the methods provided herein, where the genotype of the native nucleic acids is known but the genotype of the non-native nucleic acids is not known, the method may include a step of predicting the likely non-native genotype or determining the non-native genotype by sequencing. Further details for such methods are provided elsewhere herein such as in the Examples. In some embodiments of any one of the methods provided herein, the alleles can be determined based on prior genotyping of the native nucleic acids of the subject and/or the nucleic acids not native to the subject (e.g., of the recipient and donor, respectively). Methods for genotyping are well known in the art. Such methods include sequencing, such as next generation, hybridization, microarray, other separation technologies or PCR assays. Any one of the methods provided herein can include steps of obtaining such genotypes.

“Obtaining” as used herein refers to any method by which the respective information or materials can be acquired. Thus, the respective information can be acquired by experimental methods, such as to determine the native genotype. Respective materials can be created, designed, etc. with various experimental or laboratory methods, in some embodiments. The respective information or materials can also be acquired by being given or provided with the information, such as in a report, or materials. Materials may be given or provided through commercial means (i.e. by purchasing), in some embodiments.

Reports may be in oral, written (or hard copy) or electronic form, such as in a form that can be visualized or displayed. In some embodiments, the “raw” results for each assay as provided herein are provided in a report, and from this report, further steps can be taken to determine the amount of non-native nucleic acids in the sample. These further steps may include any one or more of the following, selecting informative results, obtaining the native and/or non-native genotype, calculating allele percentages for informative results for the native and non-native nucleic acids, averaging the allele percentages, etc. In other embodiments, the report provides the amount of non-native nucleic acids in the sample. From the amount, in some embodiments, a clinician may assess the need for a treatment for the subject or the need to monitor the amount of the non-native nucleic acids. Accordingly, in any one of the methods provided herein, the method can include assessing the amount of non-nucleic acids in the subject at another point in time. Such assessing can be performed with any one of the methods or compositions provided herein.

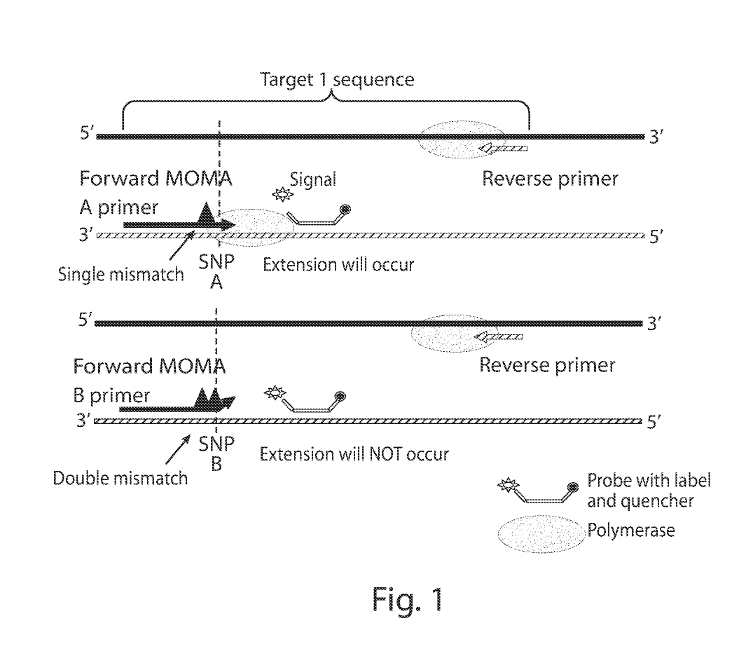

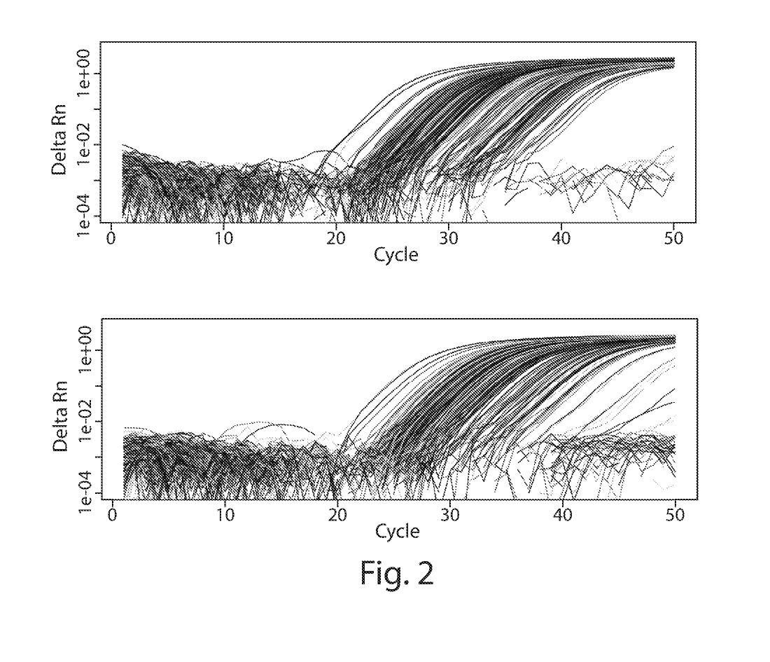

The quantitative assays as provided herein make use of multiplexed optimized mismatch amplification (MOMA). Primers for use in such assays may be obtained, and any one of the methods provided herein can include a step of obtaining one or more primer pairs for performing the quantitative assays. Generally, the primers possess unique properties that facilitate their use in quantifying amounts of nucleic acids. For example, a forward primer of a primer pair can be mismatched at a 3′ nucleotide (e.g., penultimate 3′ nucleotide). In some embodiments of any one of the methods or compositions provided, this mismatch is at a 3′ nucleotide but adjacent to the SNV position. In some embodiments of any one of the methods or composition provided, the mismatch positioning of the primer relative to a SNV position is as shown in FIG. 1. Generally, such a forward primer even with the 3′ mismatch to produce an amplification product (in conjunction with a suitable reverse primer) in an amplification reaction, thus allowing for the amplification and resulting detection of a nucleic acid with the respective SNV. If the particular SNV is not present, and there is a double mismatch with respect to the other allele of the SNV target, an amplification product will generally not be produced. Preferably, in some embodiments of any one of the methods or compositions provided herein, for each SNV target a primer pair is obtained whereby specific amplification of each allele can occur without amplification of the other allele(s). “Specific amplification” refers to the amplification of a specific allele of a target without substantial amplification of another nucleic acid or without amplification of another nucleic acid sequence above background or noise. In some embodiments, specific amplification results only in the amplification of the specific allele.

In some embodiments of any one of the methods or compositions provided herein, for each SNV target that is biallelic, there are two primer pairs, each specific to one of the two alleles and thus have a single mismatch with respect to the allele it is to amplify and a double mismatch with respect to the allele it is not to amplify (again if nucleic acids of these alleles are present). In some embodiments of any one of the methods or compositions provided herein, the mismatch primer is the forward primer. In some embodiments of any one of the methods or compositions provided herein, the reverse primer of the two primer pairs for each SNV target is the same.

These concepts can be used in the design of primer pairs for any one of the compositions and methods provided herein. It should be appreciated that the forward and reverse primers are designed to bind opposite strands (e.g., a sense strand and an antisense strand) in order to amplify a fragment of a specific locus of the template. The forward and reverse primers of a primer pair may be designed to amplify a nucleic acid fragment of any suitable size to detect the presence of, for example, an allele of a SNV target according to the disclosure. Any one of the methods provided herein can include one or more steps for obtaining one or more primer pairs as described herein.

It should be appreciated that the primer pairs described herein may be used in a multiplex PCR assay. Accordingly, in some embodiments, the primer pairs are designed to be compatible with other primer pairs in a PCR reaction. For example, the primer pairs may be designed to be compatible with at least 2, at least 5, at least 10, at least 20, at least 30, at least 40, etc. other primer pairs in a PCR reaction. As used herein, primer pairs in a PCR reaction are “compatible” if they are capable of amplifying their target in the same PCR reaction. In some embodiments, primer pairs are compatible if the primer pairs are inhibited from amplifying their target DNA by no more than 1%, no more than 2%, no more than 5%, no more than 10%, no more than 15%, no more than 20%, no more than 25%, no more than 30%, no more than 35%, no more than 40%, no more than 45%, no more than 50%, or no more than 60% when multiplexed in the same PCR reaction. Primer pairs may not be compatible for a number of reasons including, but not limited to, the formation of primer dimers and binding to off-target sites on a template that may interfere with another primer pair. Accordingly, the primer pairs of the disclosure may be designed to prevent the formation of dimers with other primer pairs or limit the number of off-target binding sites. Exemplary methods for designing primers for use in a multiplex PCR assay are known in the art and are otherwise described herein.

In some embodiments, the primer pairs described herein are used in a multiplex PCR assay to quantify an amount of non-native nucleic acids. Accordingly, in some embodiments of any one of the methods or compositions provided herein, the primer pairs are designed to detect genomic regions that are diploid, excluding primer pairs that are designed to detect genomic regions that are potentially non-diploid. In some embodiments of any one of the methods or compositions provided herein, the primer pairs used in accordance with the disclosure do not detect repeat-masked regions, known copy-number variable regions, or other genomic regions that may be non-diploid.

In some embodiments of any one of the methods provided herein, the amplification-based quantitative assay is any quantitative assay whereby nucleic acids are amplified and the amounts of the nucleic acids can be determined. Such assays include those whereby nucleic acids are amplified with the MOMA primers as described herein and quantified. Such assays include simple amplification and detection, hybridization techniques, separation technologies, such as electrophoresis, next generation sequencing and the like.

In some embodiments of any one of the methods provided herein, the quantitative assays are quantitative PCR assays. Quantitative PCR include real-time PCR, digital PCR, Taqman, etc. In some embodiments of any one of the methods provided herein the PCR is “Real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

The use of multiple probes can expand the capability of single-probe real-time PCR. Multiplex real-time PCR uses multiple probe-based assays, in which each assay has a specific probe labeled with a unique fluorescent dye, resulting in different observed colors for each assay. Real-time PCR instruments can discriminate between the fluorescence generated from different dyes. Different probes can be labeled with different dyes that each have unique emission spectra. Spectral signals are collected with discrete optics, passed through a series of filter sets, and collected by an array of detectors. Spectral overlap between dyes may be corrected by using pure dye spectra to deconvolute the experimental data by matrix algebra.

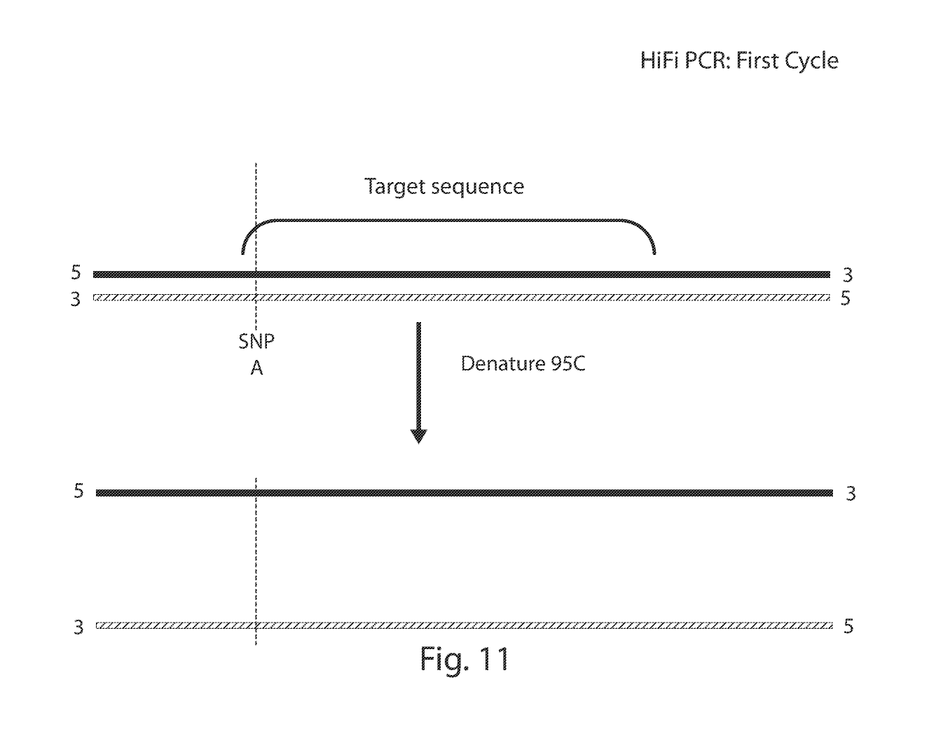

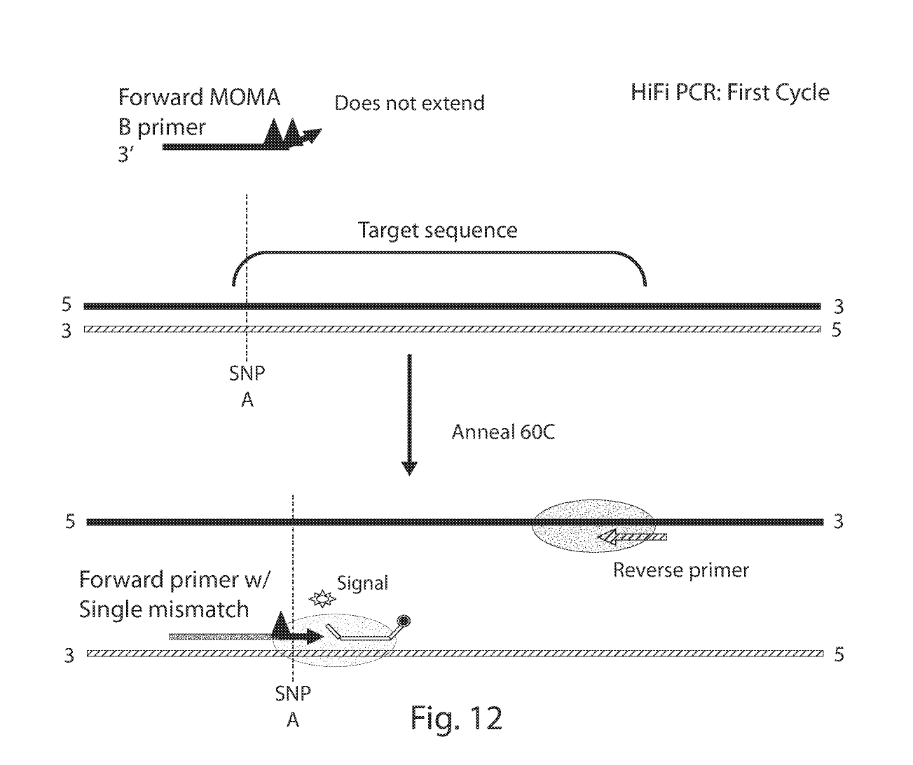

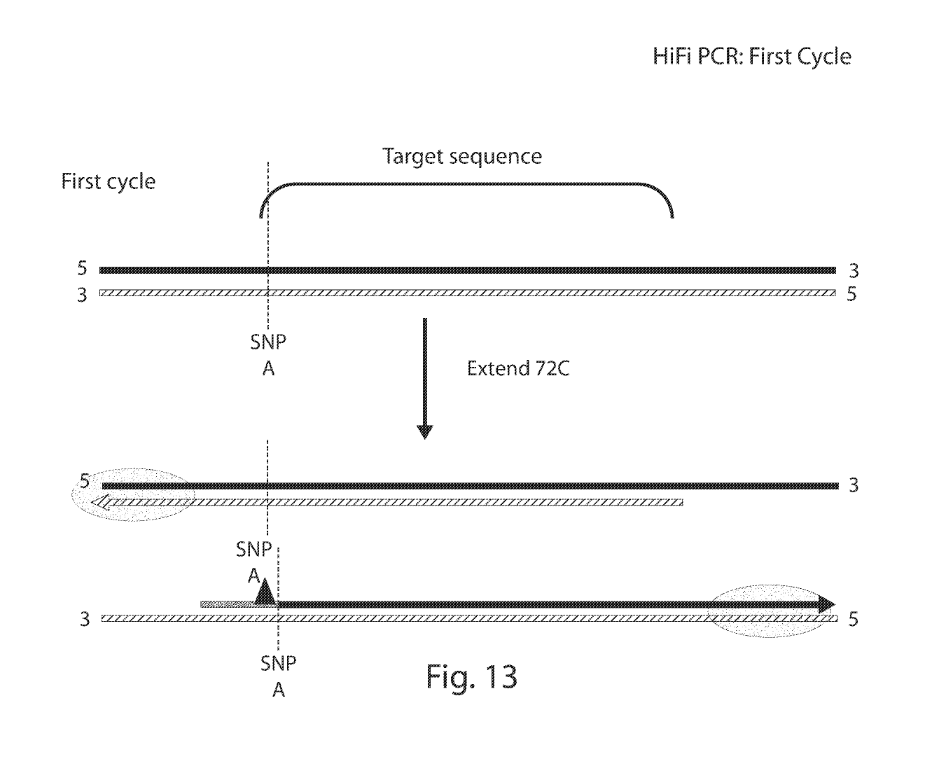

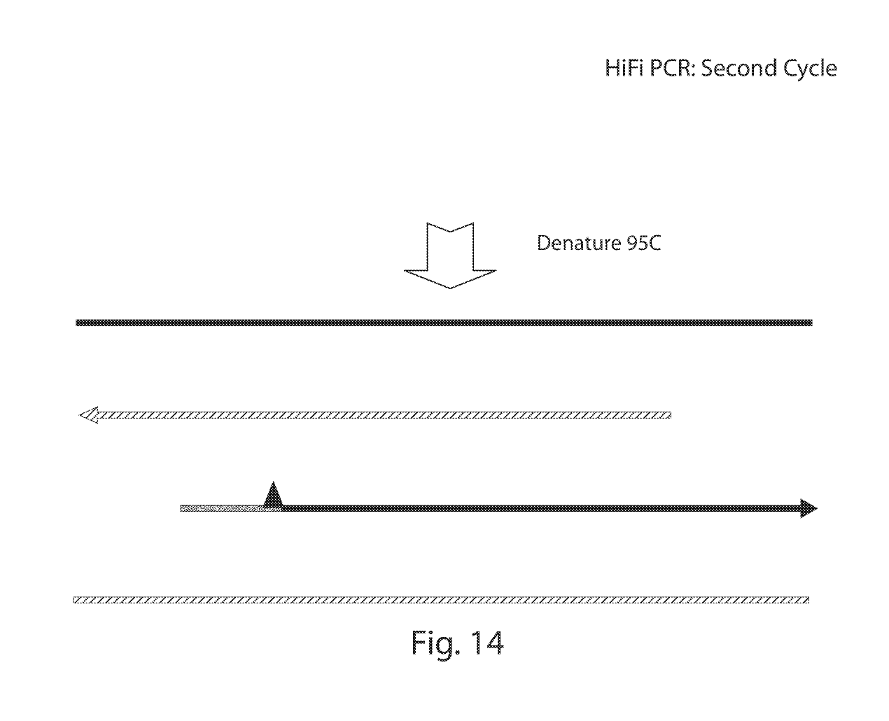

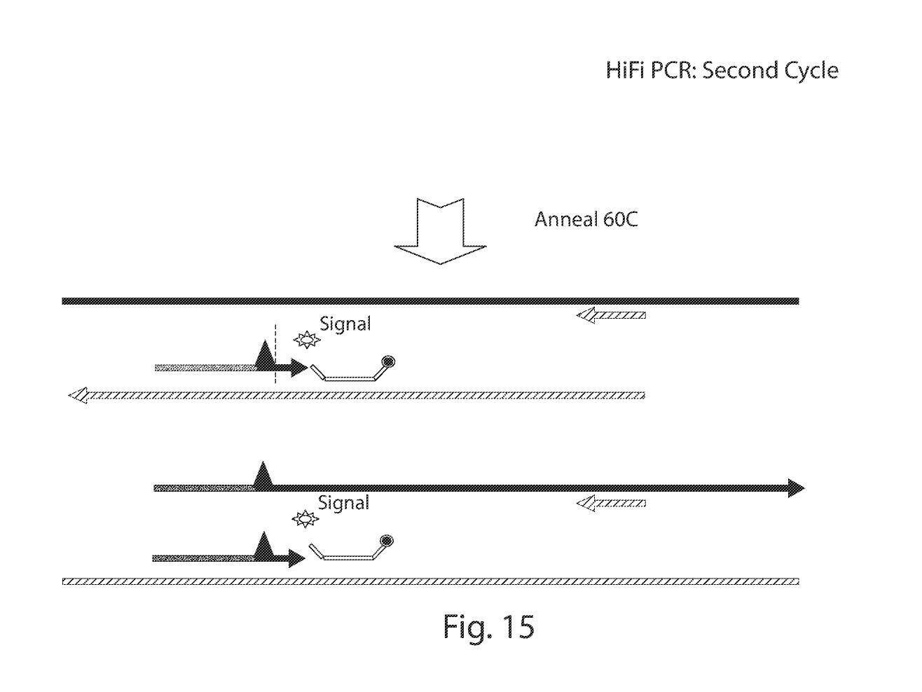

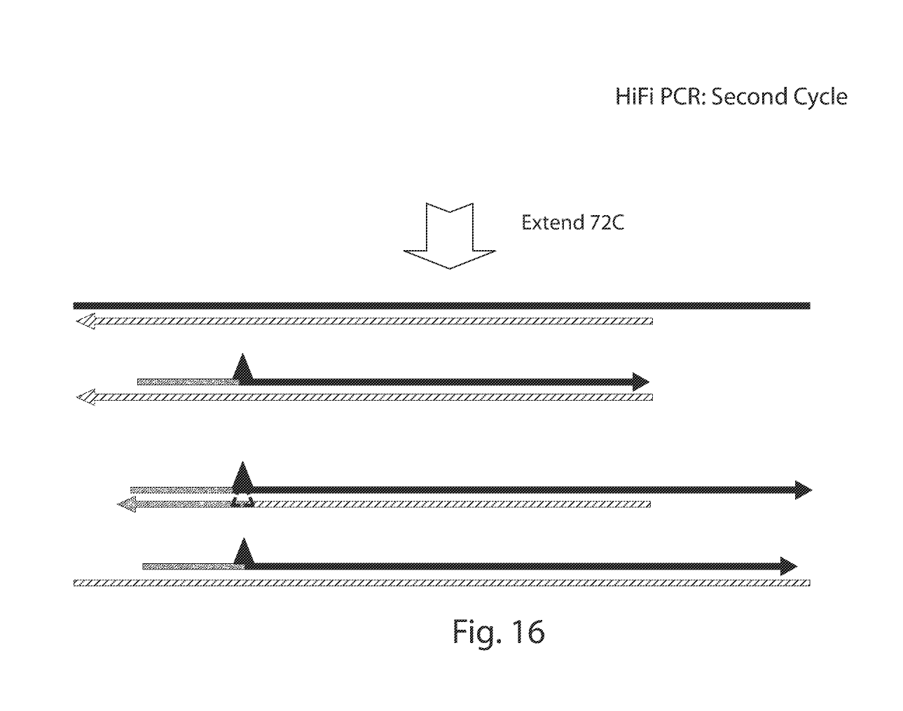

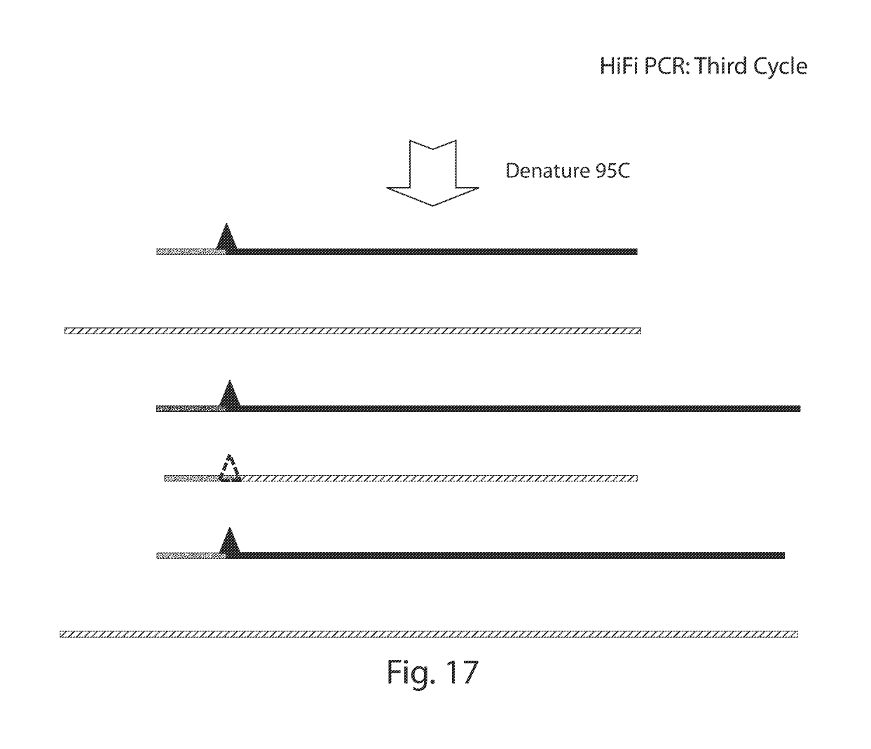

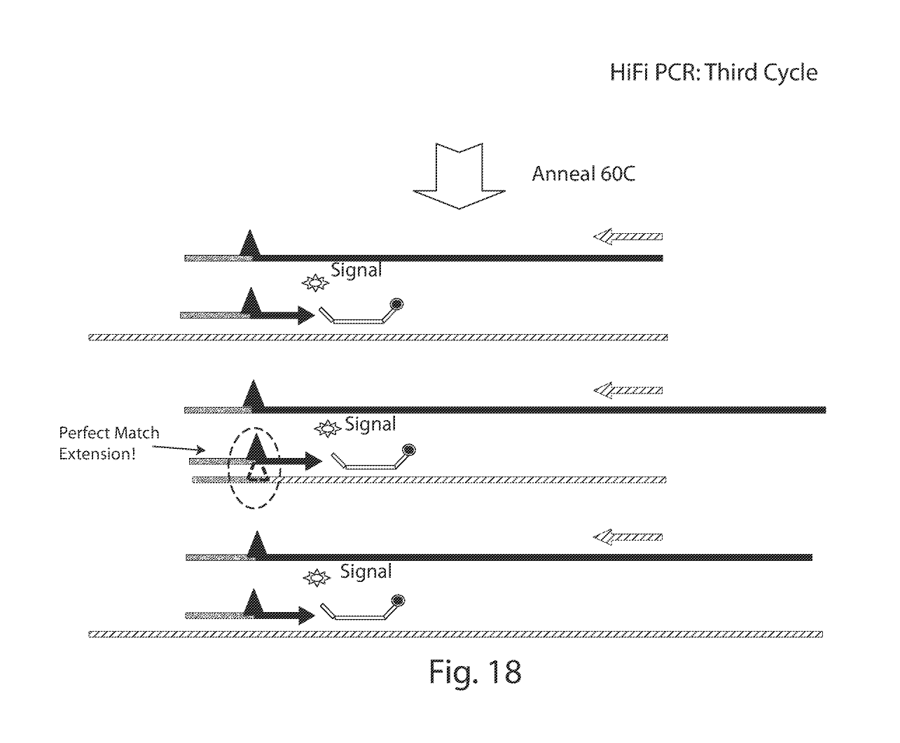

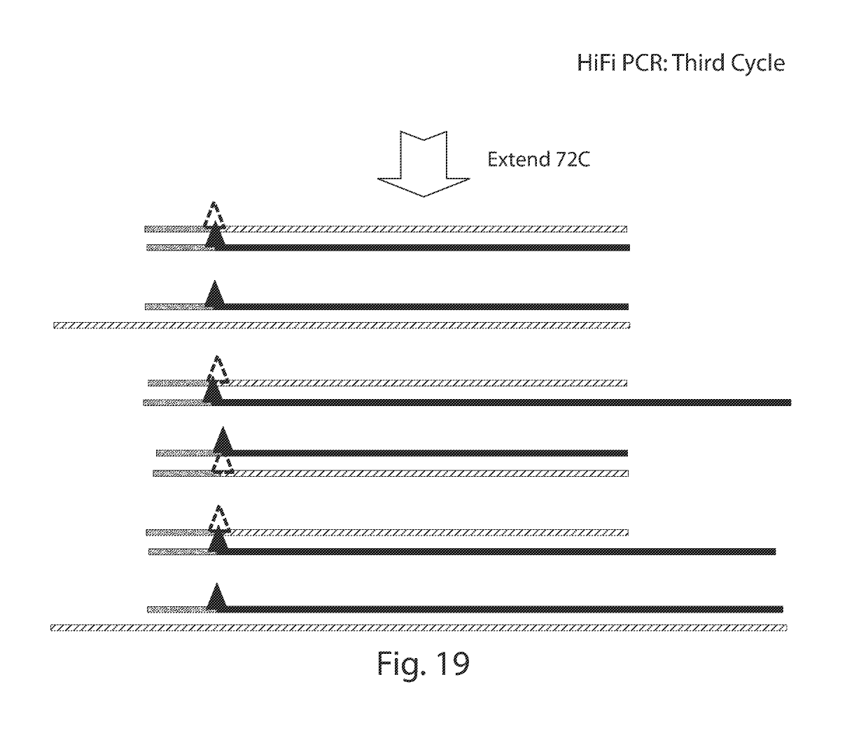

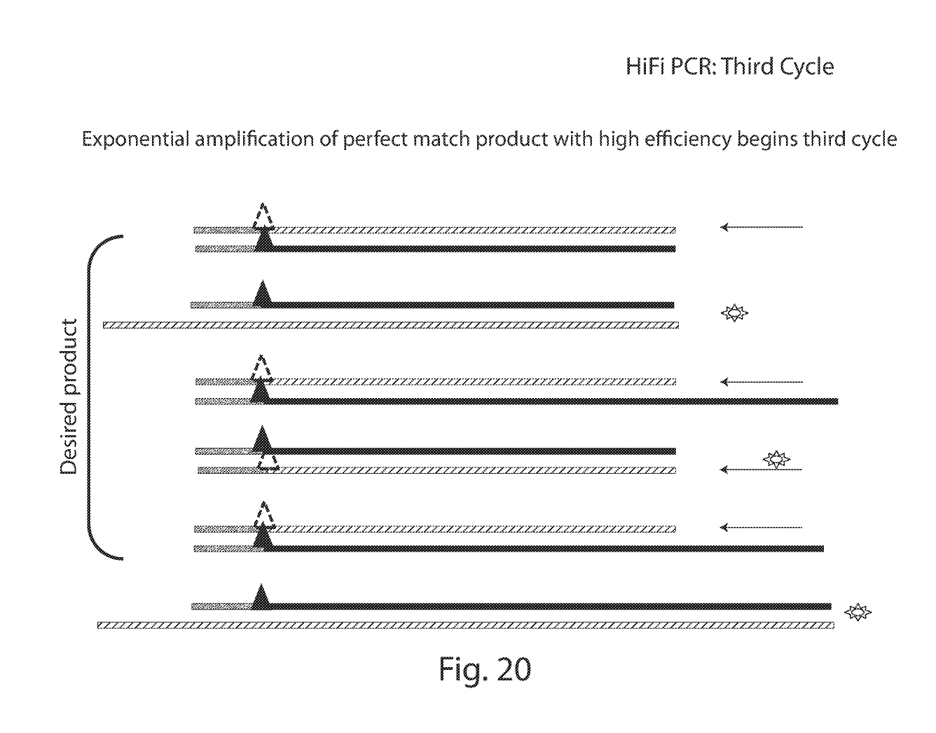

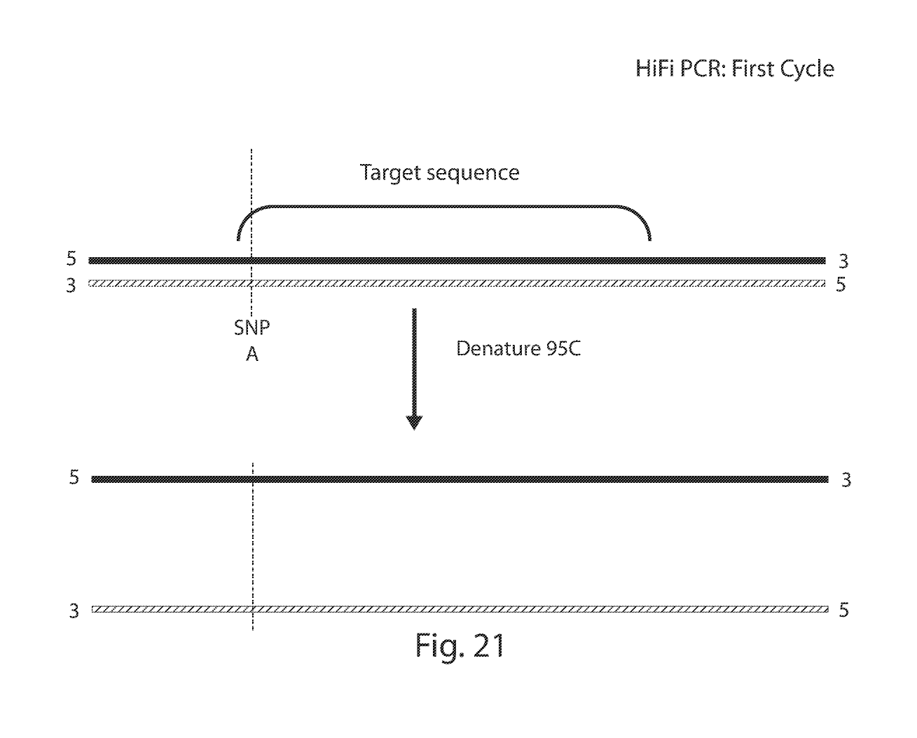

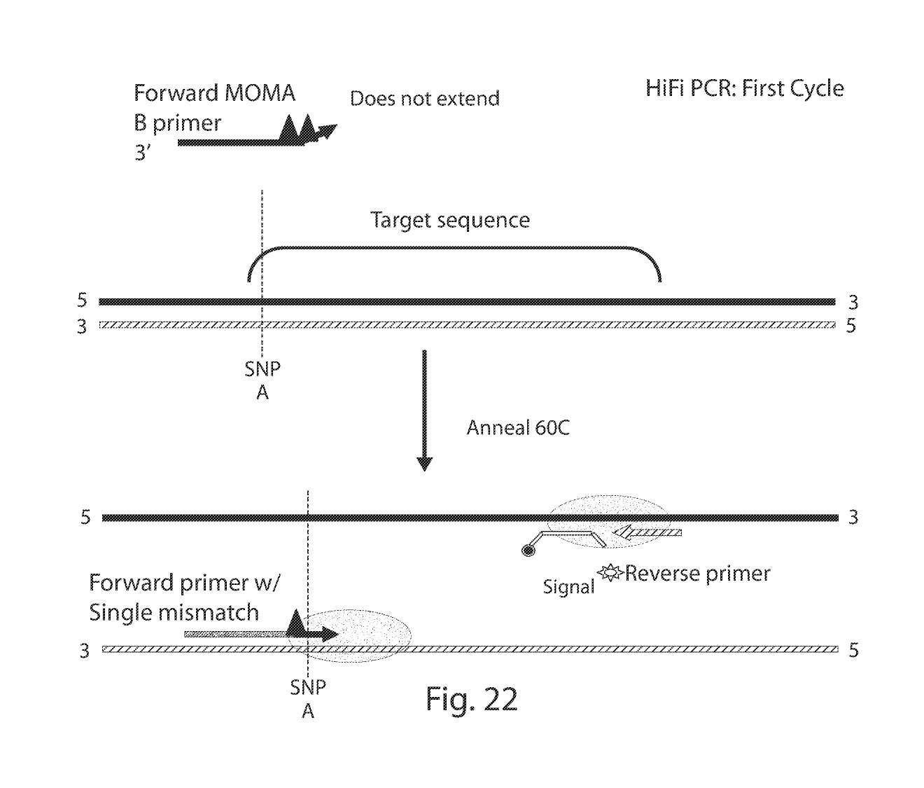

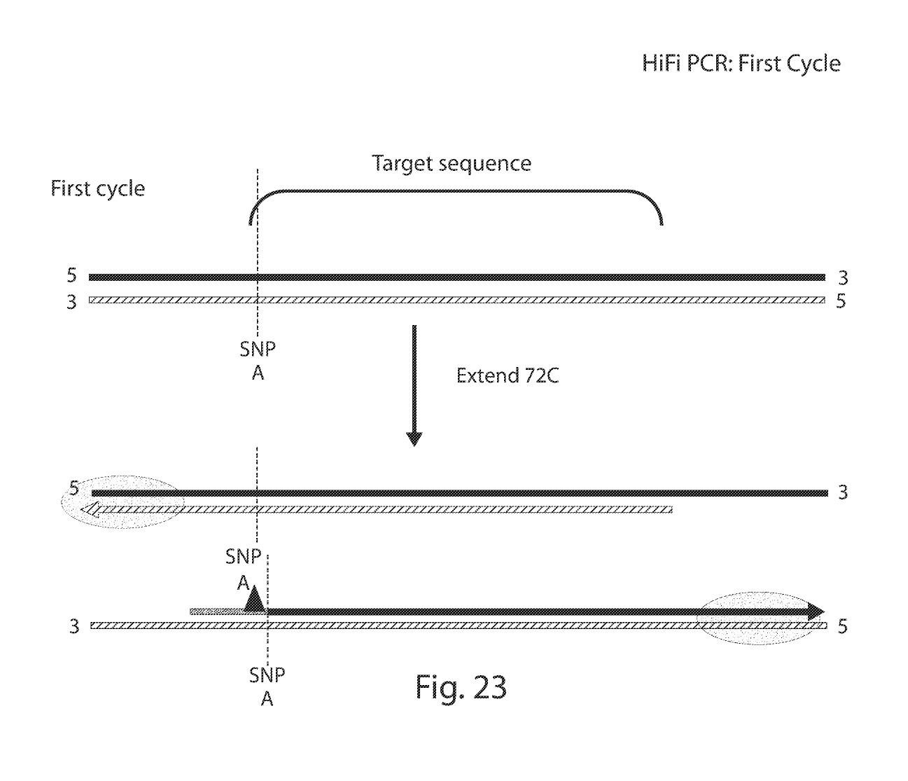

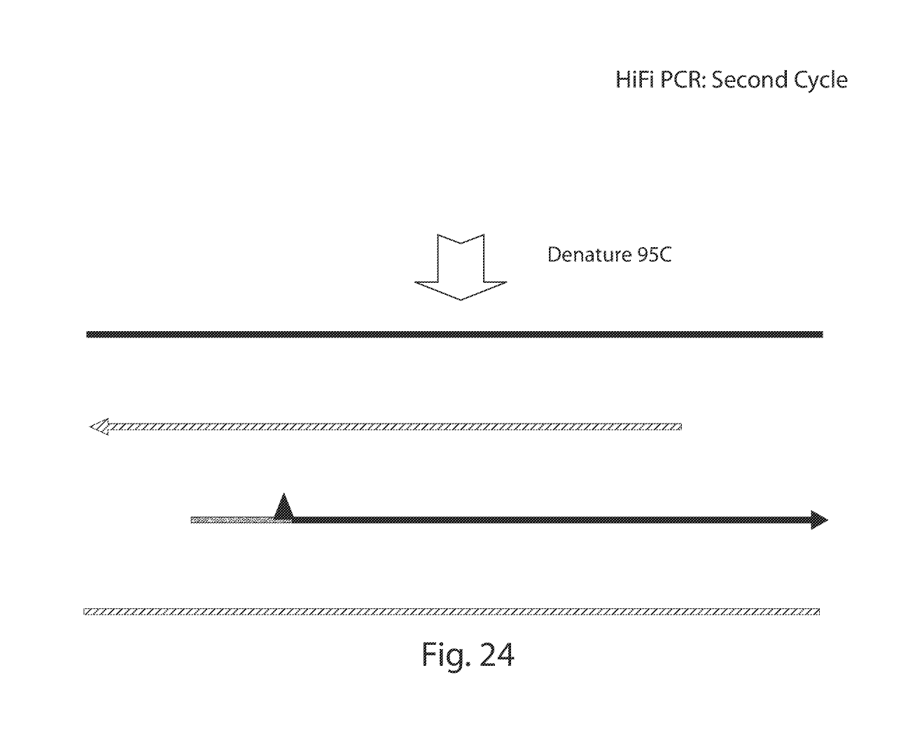

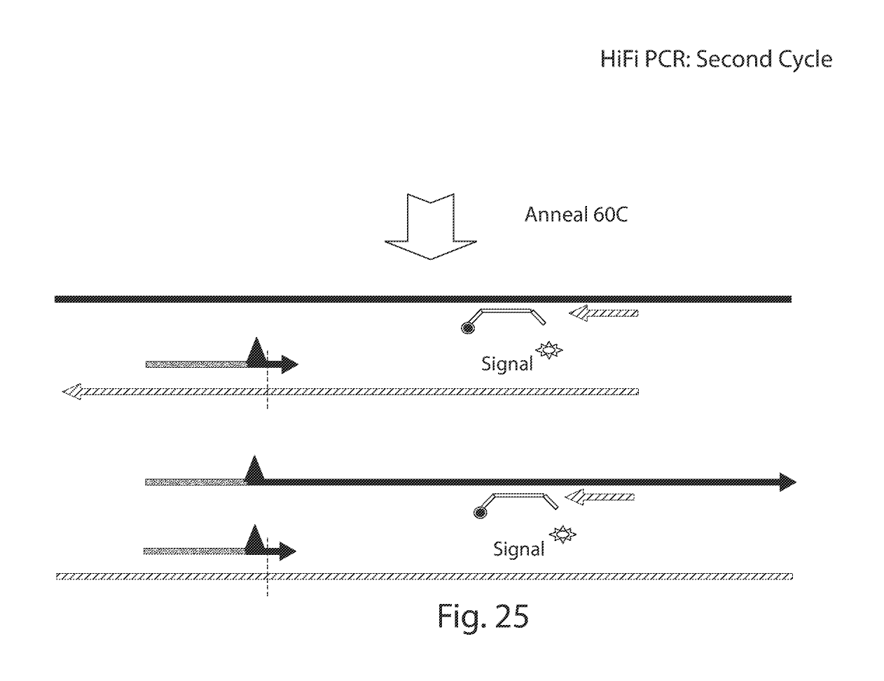

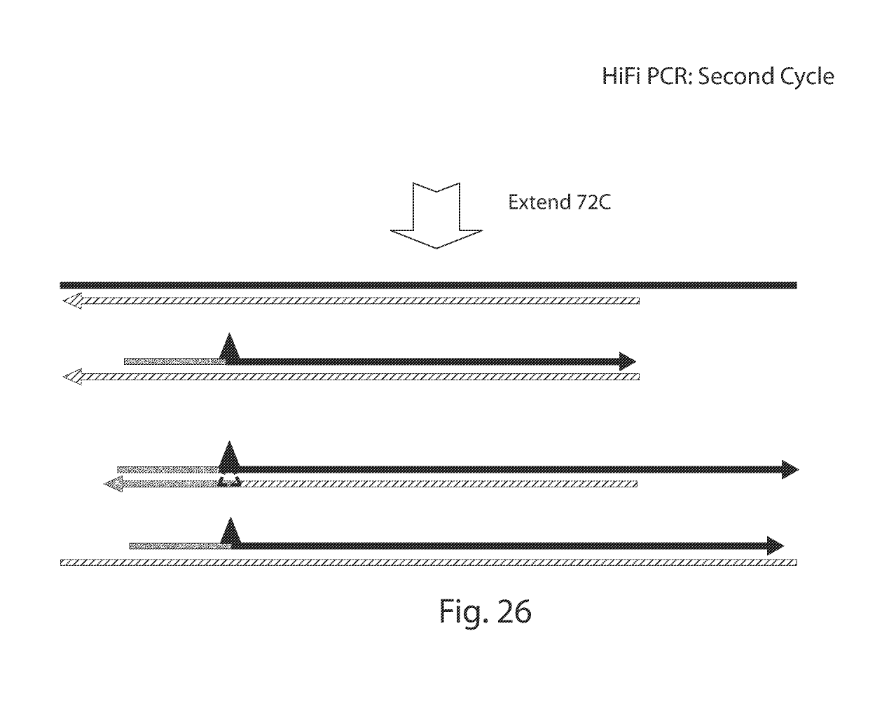

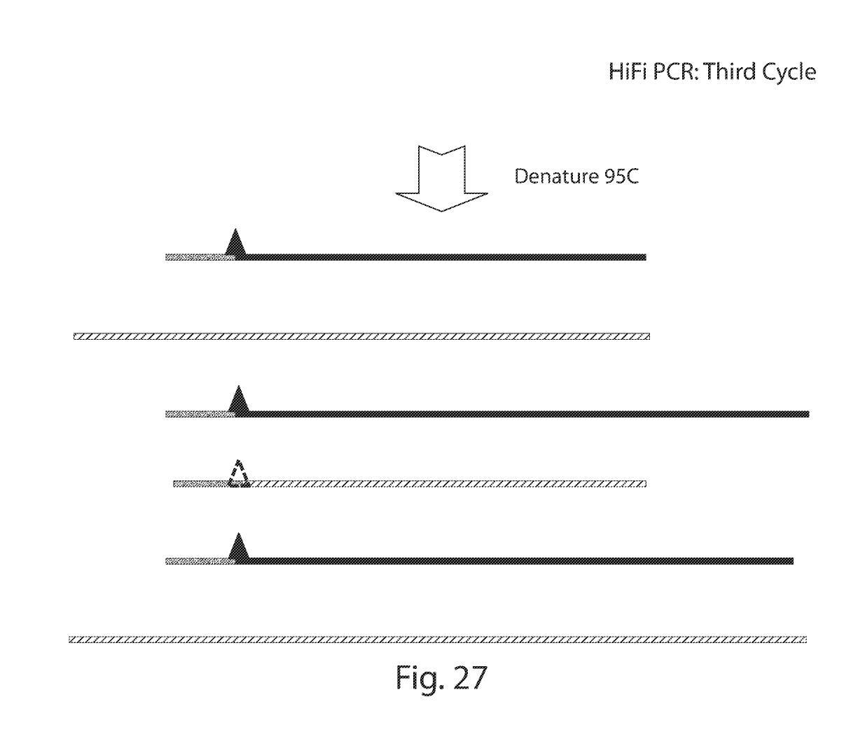

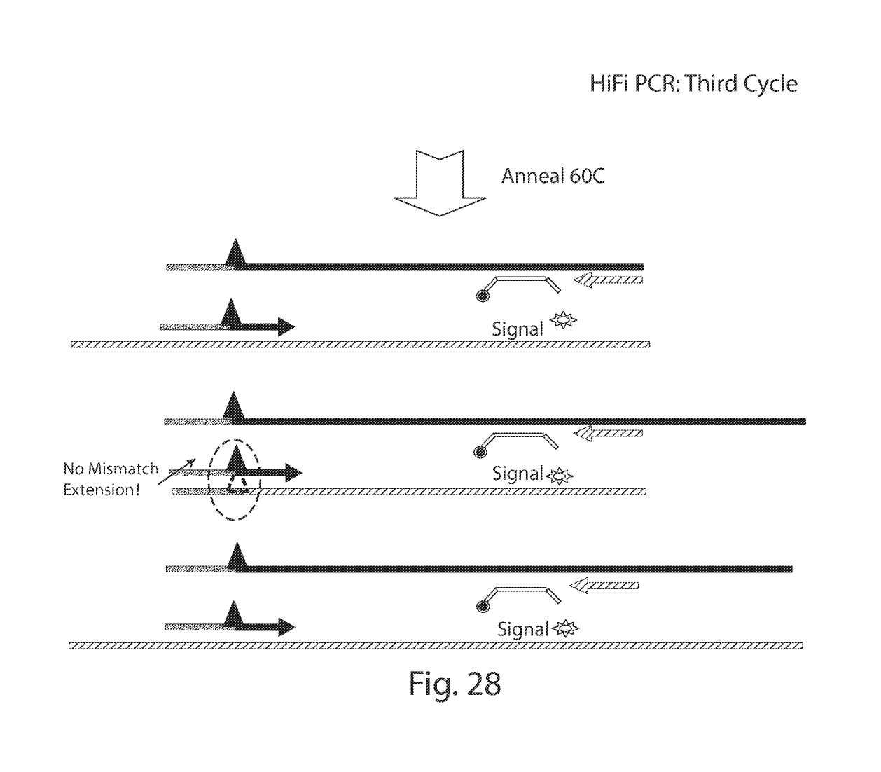

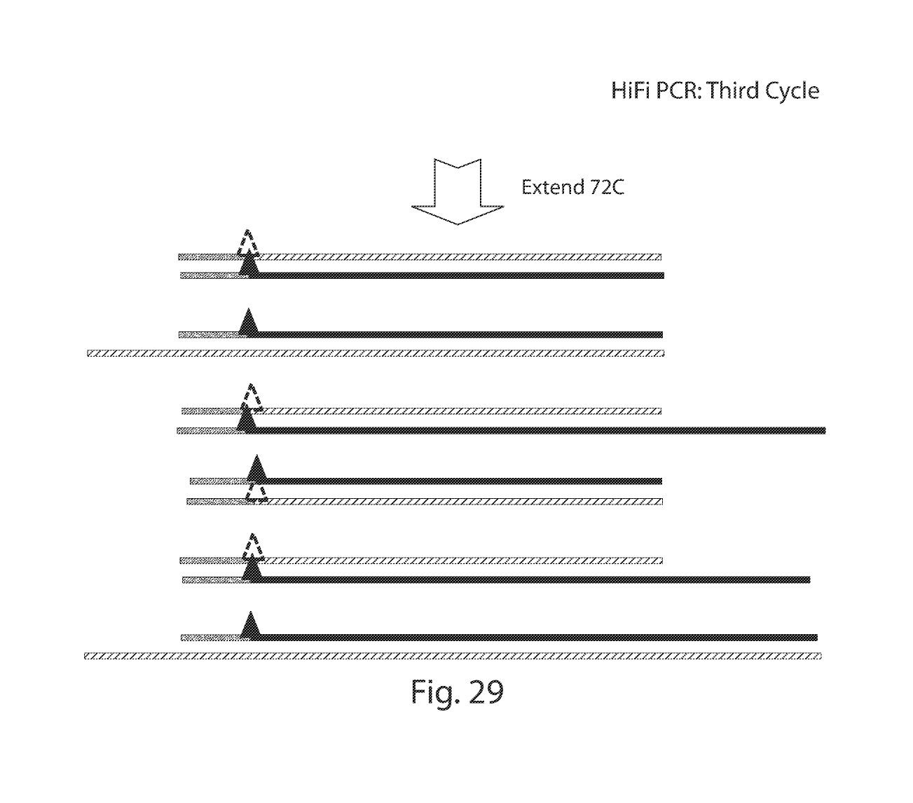

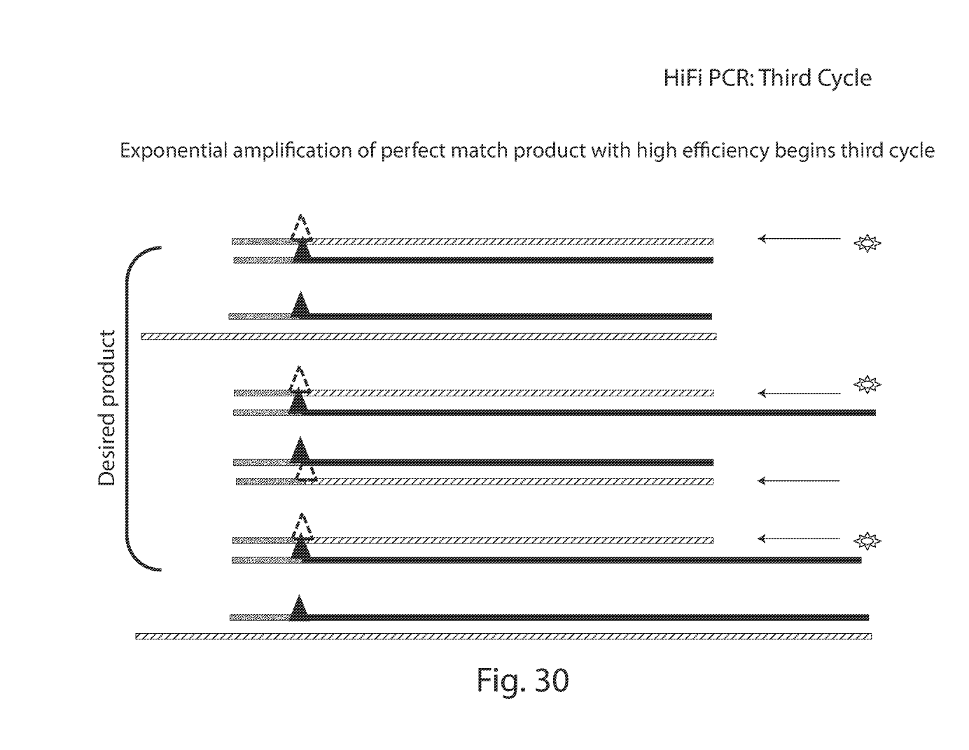

A probe may be useful for methods of the present disclosure, particularly for those methods that include a quantification step. Any one of the methods provided herein can include the use of a probe in the performance of the PCR assay(s), while any one of the compositions of kits provided herein can include one or more probes. Importantly, in some embodiments of any one of the methods provided herein, the probe in one or more or all of the PCR quantification assays is on the same strand as the mismatch primer and not on the opposite strand. It has been found that in so incorporating the probe in a PCR reaction, additional allele specific discrimination can be provided. This is illustrated in FIGS. 11-30.

As an example, a TaqMan® probe is a hydrolysis probe that has a FAM™ or VIC® dye label on the 5′ end, and minor groove binder (MGB) non-fluorescent quencher (NFQ) on the 3′ end. The TaqMan® probe principle generally relies on the 5′-3′ exonuclease activity of Taq® polymerase to cleave the dual-labeled TaqMan® probe during hybridization to a complementary probe-binding region and fluorophore-based detection. TaqMan® probes can increase the specificity of detection in quantitative measurements during the exponential stages of a quantitative PCR reaction.

PCR systems generally rely upon the detection and quantitation of fluorescent dyes or reporters, the signal of which increase in direct proportion to the amount of PCR product in a reaction. For example, in the simplest and most economical format, that reporter can be the double-strand DNA-specific dye SYBR® Green (Molecular Probes). SYBR Green is a dye that binds the minor groove of double stranded DNA. When SYBR Green dye binds to a double stranded DNA, the fluorescence intensity increases. As more double stranded amplicons are produced, SYBR Green dye signal will increase.

In any one of the methods provided herein the PCR may be digital PCR. Digital PCR involves partitioning of diluted amplification products into a plurality of discrete test sites such that most of the discrete test sites comprise either zero or one amplification product. The amplification products are then analyzed to provide a representation of the frequency of the selected genomic regions of interest in a sample. Analysis of one amplification product per discrete test site results in a binary “yes-or-no” result for each discrete test site, allowing the selected genomic regions of interest to be quantified and the relative frequency of the selected genomic regions of interest in relation to one another be determined. In certain aspects, in addition to or as an alternative, multiple analyses may be performed using amplification products corresponding to genomic regions from predetermined regions. Results from the analysis of two or more predetermined regions can be used to quantify and determine the relative frequency of the number of amplification products. Using two or more predetermined regions to determine the frequency in a sample reduces a possibility of bias through, e.g., variations in amplification efficiency, which may not be readily apparent through a single detection assay. Methods for quantifying DNA using digital PCR are known in the art and have been previously described, for example in U.S. Patent Publication number US20140242582.

It should be appreciated that the PCR conditions provided herein may be modified or optimized to work in accordance with any one of the methods described herein. Typically, the PCR conditions are based on the enzyme used, the target template, and/or the primers. In some embodiments, one or more components of the PCR reaction is modified or optimized. Non-limiting examples of the components of a PCR reaction that may be optimized include the template DNA, the primers (e.g., forward primers and reverse primers), the deoxynucleotides (dNTPs), the polymerase, the magnesium concentration, the buffer, the probe (e.g., when performing real-time PCR), the buffer, and the reaction volume.

In any of the foregoing embodiments, any DNA polymerase (enzyme that catalyzes polymerization of DNA nucleotides into a DNA strand) may be utilized, including thermostable polymerases. Suitable polymerase enzymes will be known to those skilled in the art, and include E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. Exemplary polymerases include, but are not limited to Bacillus stearothermophilus pol I, Thermus aquaticus (Taq) pol I, Pyrococcus furiosus (Pfu), Pyrococcus woesei (Pwo), Thermus flavus (Tfl), Thermus thermophilus (Tth), Thermus litoris (Tli) and Thermotoga maritime (Tma). These enzymes, modified versions of these enzymes, and combination of enzymes, are commercially available from vendors including Roche, Invitrogen, Qiagen, Stratagene, and Applied Biosystems. Representative enzymes include PHUSION® (New England Biolabs, Ipswich, Mass.), Hot MasterTaq™ (Eppendorf), PHUSION® Mpx (Finnzymes), PyroStart® (Fermentas), KOD (EMD Biosciences), Z-Taq (TAKARA), and CS3AC/LA (KlenTaq, University City, Mo.).

Salts and buffers include those familiar to those skilled in the art, including those comprising MgCl2, and Tris-HCl and KCl, respectively. Typically, 1.5-2.0 nM of magnesium is optimal for Taq DNA polymerase, however, the optimal magnesium concentration may depend on template, buffer, DNA and dNTPs as each has the potential to chelate magnesium. If the concentration of magnesium [Mg2+] is too low, a PCR product may not form. If the concentration of magnesium [Mg2+] is too high, undesired PCR products may be seen. In some embodiments the magnesium concentration may be optimized by supplementing magnesium concentration in 0.1 mM or 0.5 mM increments up to about 5 mM.

Buffers used in accordance with the disclosure may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), which are also added to a reaction adequate amount for amplification of the target nucleic acid. In some embodiments, the concentration of one or more dNTPs (e.g., dATP, dCTP, dGTP, dTTP) is from about 10 μM to about 500 μM which may depend on the length and number of PCR products produced in a PCR reaction.

In some embodiments, the primers used in accordance with the disclosure are modified. The primers may be designed to bind with high specificity to only their intended target (e.g., a particular SNV) and demonstrate high discrimination against further nucleotide sequence differences. The primers may be modified to have a particular calculated melting temperature (Tm), for example a melting temperature ranging from 46° C. to 64° C. To design primers with desired melting temperatures, the length of the primer may be varied and/or the GC content of the primer may be varied. Typically, increasing the GC content and/or the length of the primer will increase the Tm of the primer. Conversely, decreasing the GC content and/or the length of the primer will typically decrease the Tm of the primer. It should be appreciated that the primers may be modified by intentionally incorporating mismatch(es) with respect to the target in order to detect a particular SNV (or other form of sequence non-identity) over another with high sensitivity. Accordingly, the primers may be modified by incorporating one or more mismatches with respect to the specific sequence (e.g., a specific SNV) that they are designed to bind.

In some embodiments, the concentration of primers used in the PCR reaction may be modified or optimized. In some embodiments, the concentration of a primer (e.g., a forward or reverse primer) in a PCR reaction may be, for example, about 0.05 μM to about 1 μM. In particular embodiments, the concentration of each primer is about 1 nM to about 1 μM. It should be appreciated that the primers in accordance with the disclosure may be used at the same or different concentrations in a PCR reaction. For example, the forward primer of a primer pair may be used at a concentration of 0.5 μM and the reverse primer of the primer pair may be used at 0.1 μM. The concentration of the primer may be based on factors including, but not limited to, primer length, GC content, purity, mismatches with the target DNA or likelihood of forming primer dimers.

In some embodiments, the thermal profile of the PCR reaction is modified or optimized. Non-limiting examples of PCR thermal profile modifications include denaturation temperature and duration, annealing temperature and duration and extension time.

The temperature of the PCR reaction solutions may be sequentially cycled between a denaturing state, an annealing state, and an extension state for a predetermined number of cycles. The actual times and temperatures can be enzyme, primer, and target dependent. For any given reaction, denaturing states can range in certain embodiments from about 70° C. to about 100° C. In addition, the annealing temperature and time can influence the specificity and efficiency of primer binding to a particular locus within a target nucleic acid and may be important for particular PCR reactions. For any given reaction, annealing states can range in certain embodiments from about 20° C. to about 75° C. In some embodiments, the annealing state can be from about 46° C. to 64° C. In certain embodiments, the annealing state can be performed at room temperature (e.g., from about 20° C. to about 25° C.).

Extension temperature and time may also impact the allele product yield. For a given enzyme, extension states can range in certain embodiments from about 60° C. to about 75° C.

Quantification of the amounts of the alleles from a quantification assay as provided herein can be performed as provided herein or as otherwise would be apparent to one of ordinary skill in the art. As an example, amplification traces are analyzed for consistency and robust quantification. Internal standards may be used to translate the Cycle threshold to amount of input nucleic acids (e.g., DNA). The amounts of alleles can be computed as the mean of performant assays and can be adjusted for genotype. The wide range of efficient amplifications shows successful detection of low concentration nucleic acids. The percent donor can be computed as the trimmed mean of all performant assays (e.g., nanograms non-native allele to nanograms native allele ratio). Amounts can be determined with an adjustment for genotypes.

It has been found that the methods and compositions provided herein can be used to detect low-level nucleic acids, such as non-native nucleic acids, in a sample. Accordingly, the methods provided herein can be used on samples where detection of relatively rare nucleic acids is needed. In some embodiments, any one of the methods provided herein can be used on a sample to detect non-native nucleic acids that are less that 1.5% of the nucleic acids in the sample. In other embodiments, any one of the methods provided herein can be used on a sample where less than 1.3%, 1.2%, 1.1%, 1%, 0.9%, 0.8%, 0.7%, 0.6%, 0.5% 0.3%, 0.2%, 0.1%, 0.09%, 0.05%, 0.03%, or 0.01% of the nucleic acids in the sample are non-native. In other embodiments, any one of the methods provided herein can be used on a sample where at least 0.005%, 0.01%, 0.03% or 0.05% of the nucleic acids are non-native. In still other embodiments of any one of the methods provided herein, at least 0.005% but less than 1.3%, 1.2%, 1.1%, 1%, 0.9%, 0.8%, 0.7%, 0.6%, 0.5% 0.3%, 0.2%, 0.1%, 0.09%, 0.05%, 0.03%, or 0.01% of the nucleic acids in the sample are non-native.

Because of the ability to determine amounts of non-native nucleic acids, even at low levels, the methods and compositions provided herein can be used to assess a risk in a subject, such as a transplant recipient. A “risk” as provided herein, refers to the presence or absence of any undesirable condition in a subject (such as a transplant recipient), or an increased likelihood of the presence or absence of such a condition, e.g., transplant rejection. As provided herein “increased risk” refers to the presence of any undesirable condition in a subject or an increased likelihood of the presence of such a condition. As provided herein, “decreased risk” refers to the absence of any undesirable condition in a subject or a decreased likelihood of the presence (or increased likelihood of the absence) of such a condition.

As an example, early detection of rejection following implantation of a transplant (e.g., a heart transplant) can facilitate treatment and improve clinical outcomes. Transplant rejection remains a major cause of graft failure and late mortality and generally requires lifelong surveillance monitoring. Treatment of transplant rejections with immunosuppressive therapy has been shown to improve treatment outcomes, particularly if rejection is detected early. Transplant rejection is typically monitored using a catheter-based endomyocardial biopsy (EMB). This invasive procedure, however, is associated with risks and discomfort for a patient, and may be particularly disadvantageous for pediatric patients. Accordingly, provided herein are sensitive, specific, cost effective, and non-invasive techniques for the surveillance of subjects, such as transplant recipients. Such techniques have been found to allow for the detection of transplant rejection at an early stage. Such techniques can also be used to monitor organ recovery and in the selection and monitoring of a treatment or therapy, such as an anti-rejection treatment, thus improving a patient's recovery and increasing survival rates.

Accordingly, in some embodiments of any one of the methods provided, the subject is a recipient of a transplant, and the risk is a risk associated with the transplant. In some embodiments of any one of the methods provided, the risk associated with the transplant is risk of transplant rejection, an anatomical problem with the transplant or injury to the transplant. In some embodiments of any one of the methods provided, the injury to the transplant is initial or ongoing injury. In some embodiments of any one of the methods provided, the risk associated with the transplant is an acute condition or a chronic condition. In some embodiments of any one of the methods provided, the acute condition is transplant rejection including cellular rejection or antibody mediate rejection. In some embodiments of any one of the methods provided, the chronic condition is graft vasculopathy. In some embodiments of any one of the methods provided, the risk associated with the transplant is indicative of the severity of the injury.

As used herein, “transplant” refers to the moving of an organ from a donor to a recipient for the purpose of replacing the recipient's damaged or absent organ. The transplant may be of one organ or more than one organ. In some embodiments, the term “transplant” refers to a transplanted organ or organs, and such meaning will be clear from the context the term is used. Examples of organs that can be transplanted include, but are not limited to, the heart, kidney(s), kidney, liver, lung(s), pancreas, intestine etc. Any one of the methods or compositions provided herein may be used on a sample from a subject that has undergone a transplant of any one or more of the organs provided herein. In some embodiments, the transplant is a heart transplant.

The risk in a recipient of a transplant can be determined, for example, by assessing the amount of non-native cf-DNA, such as donor-specific cell-free-DNA (DS cf-DNA), a biomarker for cellular injury related to transplant rejection. DS cf-DNA refers to DNA that presumably is shed from the transplanted organ, the sequence of which matches (in whole or in part) the genotype of the donor who donated the transplanted organ. As used herein, DS cf-DNA may refer to certain sequence(s) in the DS cf-DNA population, where the sequence is distinguishable from the recipient cf-DNA (e.g., having a different sequence at a particular nucleotide location(s)), or it may refer to the entire DS cf-DNA population.

The risk in a recipient of a transplant can be determined, for example, by assessing the amount of non-native cf-DNA, such as donor-specific cell-free DNA, as described herein using any one of the methods provided in combination with an assessment of the amount of total cell-free DNA, such as in ng/ml plasma. Thus, any one of the methods provided herein can include a step of obtaining the level of total cell-free DNA, such as in ng/ml in the subject. Such methods, in some embodiments, further includes assessing a risk associate with the transplant in the subject based on the combination of the amount of donor-specific cell-free DNA and total cell-free DNA in the subject. Methods for determining total cell-free DNA in the subject are known in the art. In some embodiments of any one of the methods provided herein, the total cell-free DNA is determined with Taqman Real-time PCR using RNase P as a target.

In some embodiments, any one of the methods provided herein can comprise correlating an increase in non-native nucleic acids and/or an increase in the ratio, or percentage, of non-native nucleic acids relative to native nucleic acids, with an increased risk of a condition, such as transplant rejection. In some embodiments of any one of the methods provided herein, correlating comprises comparing a level (e.g., concentration, ratio or percentage) of non-native nucleic acids to a threshold value to identify a subject at increased or decreased risk of a condition. In some embodiments of any one of the methods provided herein, a subject having an increased amount of non-native nucleic acids compared to a threshold value is identified as being at increased risk of a condition. In some embodiments of any one of the methods provided herein, a subject having a decreased or similar amount of non-native nucleic acids compared to a threshold value is identified as being at decreased risk of a condition.

As used herein, “amount” refers to any quantitative value for the measurement of nucleic acids and can be given in an absolute or relative amount. Further, the amount can be a total amount, frequency, ratio, percentage, etc. As used herein, the term “level” can be used instead of “amount” but is intended to refer to the same types of values.

“Threshold” or “threshold value”, as used herein, refers to any predetermined level or range of levels that is indicative of the presence or absence of a condition or the presence or absence of a risk. The threshold value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. The threshold value can depend upon the particular population selected. For example, an apparently healthy population will have a different ‘normal’ range. As another example, a threshold value can be determined from baseline values before the presence of a condition or risk or after a course of treatment. Such a baseline can be indicative of a normal or other state in the subject not correlated with the risk or condition that is being tested for. In some embodiments, the threshold value can be a baseline value of the subject being tested. Accordingly, the predetermined values selected may take into account the category in which the subject falls. Appropriate ranges and categories can be selected with no more than routine experimentation by those of ordinary skill in the art.

Changes in the levels of non-native nucleic acids can also be monitored over time. For example, a change from a threshold value (such as a baseline) in the amount, such as ratio or percentage, of non-native nucleic acids can be used as a non-invasive clinical indicator of risk, e.g., risk associated with transplant. This can allow for the measurement of variations in a clinical state and/or permit calculation of normal values or baseline levels. In organ transplantation, this can form the basis of an individualized non-invasive screening test for rejection or a risk of a condition associated thereto. Generally, as provided herein, the amount, such as the ratio or percent, of non-native nucleic acids can be indicative of the presence or absence of a risk associated with a condition, such as risk associated with a transplant, such as rejection, in the recipient, or can be indicative of the need for further testing or surveillance. In some embodiments, for transplant recipients, this amount in combination with the total amount of cell-free DNA is indicative of the risk. In one embodiment of any one of the methods provided herein, the method may further include an additional test(s) for assessing a condition, such as transplant rejection, transplant injury, etc. The additional test(s) may be any one of the methods provided herein. In some embodiments, for transplant recipients, the additional test is a determination of the amount of total cell-free DNA in a sample from the subject.

In some embodiments of any one of the methods provided herein in regard to a heart transplant recipient, such threshold is 1%, wherein a level above 1% is indicative of an increased risk and wherein a level at or below 1% is indicative of a decreased risk. In some embodiments of any one of the methods provided herein in regard to a heart transplant recipient, such threshold is 1.3%, wherein a level above 1.3% is indicative of an increased risk and wherein a level at or below 1.3% is indicative of a decreased risk.

In some embodiments of any one of the methods provided herein, where a non-native nucleic acid amount, such as ratio or percentage, is determined to be above a threshold value, any one of the methods provided herein can further comprise performing another test on the subject or sample therefrom. Such other tests can be any other test known by one of ordinary skill in the art to be useful in determining the presence or absence of a risk, e.g., in a transplant recipient. In some embodiments, the other test is any one of the methods provided herein. In some embodiments of any one of the methods provided herein, the subject is a transplant recipient and the other test is a determination of the level of BNP and/or troponin in the transplant recipient. In other embodiments of any one of the methods provided herein, the other test in addition to the level of BNP and/or troponin or in place thereof is an echocardiogram.

In some embodiments of any one of the methods provided herein, where the non-native nucleic acid amount, such as the ratio or percentage, is determined to be less than a threshold value such as 1% or 1.3% no further testing is needed or recommended to the subject and/or no treatment is needed or suggested to the subject. While in some embodiments of any one of the methods provided herein, it may be determined that there is an increased risk in the recipient when the amount of the non-native nucleic acid (e.g., ratio or percentage) in a sample obtained from the recipient is greater than 1% or 1.3%, although it should be appreciated that other thresholds may be utilized as embodiments of the invention are not limited in this respect. In some embodiments, the method may further comprise further testing or recommending further testing to the subject and/or treating or suggesting treatment to the subject. In some of these embodiments, the further testing is any one of the methods provided herein. In some of these embodiments, the treating is an anti-rejection treatment. In some embodiments, the information is provided in written form or electronic form. In some embodiments, the information may be provided as computer-readable instructions.

Anti-rejection therapies include, for example, the administration of an immunosuppressive to a transplant recipient. Immunosuppressives include, but are not limited to, corticosteroids (e.g., prednisolone or hydrocortisone), glucocorticoids, cytostatics, alkylating agents (e.g., nitrogen mustards (cyclophosphamide), nitrosoureas, platinum compounds, cyclophosphamide (Cytoxan)), antimetabolites (e.g., folic acid analogues, such as methotrexate, purine analogues, such as azathioprine and mercaptopurine, pyrimidine analogues, and protein synthesis inhibitors), cytotoxic antibiotics (e.g., dactinomycin, anthracyclines, mitomycin C, bleomycin, mithramycin), antibodies (e.g., anti-CD20, anti-IL-1, anti-IL-2Ralpha, anti-T-cell or anti-CD-3 monoclonals and polyclonals, such as Atgam, and Thymoglobuline), drugs acting on immunophilins, ciclosporin, tacrolimus, sirolimus, interferons, opiods, TNF-binding proteins, mycophenolate, fingolimod and myriocin. In some embodiments, anti-rejection therapy comprises blood transfer or marrow transplant. Therapies can also include therapies for treating systemic conditions, such as sepsis. The therapy for sepsis can include intravenous fluids, antibiotics, surgical drainage, early goal directed therapy (EGDT), vasopressors, steroids, activated protein C, drotrecogin alfa (activated), oxygen and appropriate support for organ dysfunction. This may include hemodialysis in kidney failure, mechanical ventilation in pulmonary dysfunction, transfusion of blood products, and drug and fluid therapy for circulatory failure. Ensuring adequate nutrition—preferably by enteral feeding, but if necessary by parenteral nutrition—can also be included particularly during prolonged illness. Other associated therapies can include insulin and medication to prevent deep vein thrombosis and gastric ulcers. Therapies for treating a recipient of a transplant can also include therapies for treating a bacterial, fungal and/or viral infection. Such therapies are known to those of ordinary skill in the art.

Any one of the methods provided herein can comprise extracting nucleic acids, such as cell-free DNA, from a sample obtained from a subject, such as a recipient of a transplant. Such extraction can be done using any method known in the art or as otherwise provided herein (see, e.g., Current Protocols in Molecular Biology, latest edition, or the QIAamp circulating nucleic acid kit or other appropriate commercially available kits). An exemplary method for isolating cell-free DNA from blood is described. Blood containing an anti-coagulant such as EDTA or DTA is collected from a subject. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 1317-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu R W K, Poon L L M, Lau T K, Leung T N, Wong E M C, Lo Y M D. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

As used herein, the sample from a subject can be a biological sample. Examples of such biological samples include whole blood, plasma, serum, urine, etc. In some embodiments of any one of the methods provided herein, addition of further nucleic acids, e.g., a standard, to the sample can be performed.

In some embodiments of any one of the methods provided herein, an amplification step is performed. An exemplary method of amplification is as follows, and such a method can be included in any one of the methods provided herein. ˜15 ng of cell free plasma DNA is amplified in a PCR using Q5 DNA polymerase with approximately ˜100 targets where pooled primers were at 6 uM total. Samples undergo approximately 35 cycles. Reactions are in 25 ul total. After amplification, samples can be cleaned up using several approaches including AMPURE bead cleanup, bead purification, or simply Exosap it, or Zymo. Such an amplification step was used in some methods as provided herein.

The present disclosure also provides methods for determining a plurality of SNV targets for use in any one of the methods provided herein or from which any one of the compositions of primers can be derived. A method of determining a plurality of SNV targets, in some embodiments comprises a) identifying a plurality of highly heterozygous SNVs in a population of individuals, b) designing one or more primers spanning each SNV, c) selecting sufficiently specific primers, d) evaluating multiplexing capabilities of primers, such as at a common melting temperature and/or in a common solution, and e) identifying sequences that are evenly amplified with the primers or a subset thereof.

As used herein, “highly heterozygous SNVs” are those with a minor allele at a sufficiently high percentage in a population. In some embodiments, the minor allele is at least 25%, 26%, 27%, 28%, 29%, 30%, 31%, 32%, 33%, 34% or 35% or more in the population. In any one of these embodiments, the minor allele is less than 50%, 49%, 45% or 40% in the population. Such SNVs increase the likelihood of providing a target that is different between the native and non-native nucleic acids.

Primers were designed to generally span a 70 bp window but some other window may also be selected, such as one between 60 bps and 80 bps. Also, generally, it was desired for the SNV to fall about in the middle of this window. For example, for a 70 bp window, the SNV was between bases 20-50, such as between bases 30-40. The primers as provided herein were designed to be adjacent to the SNV.

As used herein, “sufficiently specific primers”, were those that demonstrated discrimination between amplification of the intended allele versus amplification of the unintended allele. Thus, with PCR a cycle gap was desired between amplification of the two. In one embodiment, the cycle gap was at least a 5, 6, 7 or 8 cycle gap.

Further, sequences were selected based on melting temperatures, generally those with a melting temperature of between 45-55 degress C. were selected as “moderate range sequences”. Other temperature ranges may be desired and can be determined by one of ordinary skill in the art. A “moderate range sequence” generally is one that can be amplified in a multiplex amplification format within the temperature. In some embodiments, the gc % content was between 30-70%, such as between 33-66%.

In one embodiment of any one of the methods provided herein, the method can further comprise excluding sequences associated with difficult regions. “Difficult regions” are any regions with content or features that make it difficult to reliably make predictions about a target sequence or are thought to not be suitable for multiplex amplification. Such regions include syndromic regions, low complexity regions, regions with high GC content or that have sequential tandem repeats. Other such features can be determined or are otherwise known to those of ordinary skill in the art.

The present disclosure also provides compositions or kits that can be useful for assessing an amount of non-native nucleic acids in a sample. In some embodiments, the composition or kit comprises a plurality of primer pairs. Each of the primer pairs of the composition or kit can comprise a forward and a reverse primer, wherein there is a 3′ mismatch in one of the primers (e.g., at the penultimate 3′ nucleotide) in some embodiments of any one of the methods, compositions or kits provided herein. In some embodiments of any one of the methods, compositions or kits provided herein, this mismatch is at a 3′ nucleotide and adjacent to the SNV position and when the particular SNV is not present there is a double mismatch with respect to the other allele of the SNV target. In some embodiments of any one of the methods, compositions or kits provided herein, the mismatch primer of a primer pair is the forward primer. In some embodiments of any one of the methods, compositions or kits provided herein, the reverse primer for each allele of a SNV target is the same.

In some embodiments of any one of the methods, compositions or kits provided herein, there are at least 2, at least 5, at least 10, at least 20, at least 30, at least 40, etc. such primer pairs. In some embodiments of any one of the methods, compositions or kits provided, there is a primer pair, such as at least two primer pairs, for at least 40, 45, 50, 55, 60, 65, 70, 75, 80, 85, 90, 91, 92, 93, 94, 95 or more targets. In some embodiments of any one of the methods, compositions or kits provided, there is a primer pair, such as at least two primer pairs, for fewer than 105, 104, 103, 102, 101, 100, 99, 98 or 97 targets. In some embodiments of any one of the methods, compositions or kits provided, there is a primer pair, such as at least two primer pairs, for between 40-105, 45-105, 50-105, 55-105, 60-105, 65-105, 70-105, 75-105, 80-105, 85-105, 90-105, 90-104, 90-103, 90-102, 90-101, 90-100, 90-99, 91-99, 92-99, 93, 99, 94-99, 95-99, or 90-95 targets. In some embodiments of any one of the methods, compositions or kits provided, there is a primer pair, such as at least two primer pairs, for between 40-99, 45-99, 50-99, 55-99, 60-99, 65-99, 70-99, 75-99, 80-99, 85-99, 90-99, 90-99, 90-98, 90-97 or 90-96 targets. In some embodiments of any one of the methods, compositions or kits provided, there is a primer pair, such as at least two primer pairs, for between 90-105, 90-104, 90-103, 90-102, 90-101, 90-100, 90-99, 91-99, 92-99, 93, 99, 94-99, 95-99, 90-95 targets. In some embodiments of any one of the methods, compositions or kits provided herein, the primer pairs are designed to be compatible for use in a quantitative assay as provided herein. For example, the primer pairs are designed to prevent primer dimers and/or limit the number of off-target binding sites. It should be appreciated that the plurality of primer pairs of any one of the methods, compositions or kits provided may be optimized or designed in accordance with any one of the methods described herein.

In some embodiments, any one of the compositions or kits provided further comprises a buffer. In some embodiments, the buffers contain additives such as surfactants, dimethyl sulfoxide (DMSO), glycerol, bovine serum albumin (BSA) and polyethylene glycol (PEG) or other PCR reaction additive. In some embodiments, any one of the compositions or kits provided further comprises a polymerase for example, the composition or kit may comprise E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. In some embodiments, any one of the compositions or kits provided further comprises one or more dNTPs (e.g., dATP, dCTP, dGTP, dTTP). In some embodiments, any one of the compositions or kits provided further comprises a probe (e.g., a TaqMan® probe).

A “kit,” as used herein, typically defines a package or an assembly including one or more of the compositions of the invention, and/or other compositions associated with the invention, for example, as previously described. Any one of the kits provided herein may further comprise at least one reaction tube, well, chamber, or the like. Any one of the primers, primer systems (such as a set of primers for a plurality of targets) or primer compositions described herein may be provided in the form of a kit or comprised within a kit.

Each of the compositions of the kit may be provided in liquid form (e.g., in solution), in solid form (e.g., a dried powder), etc. A kit may, in some cases, include instructions in any form that are provided in connection with the compositions of the invention in such a manner that one of ordinary skill in the art would recognize that the instructions are to be associated with the compositions of the invention. The instructions may include instructions for performing any one of the methods provided herein. The instructions may include instructions for the use, modification, mixing, diluting, preserving, administering, assembly, storage, packaging, and/or preparation of the compositions and/or other compositions associated with the kit. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1

### With Recipient and Donor Genotype Information

### SNV Target Selection

Identification of suitable and compatible targets for multiplexing in accordance with the disclosure may include one or more of the following steps, exemplified below:
Begin with highly heterozygous SNPs
   Screened on several ethnic control populations
   Hardy-Weinberg p>0.25
   Excluding known difficult regions
      Syndromic regions likely abnormal in patients
      Low complexity regions, including centromeres and telomeres of chromosomes
Target fragments of desired length designed in silico
   Two 20-26 bp primers spanning each SNP's 70 bp window
   All candidate primers queried with BLAST to GCRh37
   Sufficiently specific primers retained
      Monitoring for off target hits particularly at the 3′ end of the fragment
      Analyzed off-target candidate hits for pairwise fragment generation that would survive size selection
Multiplexing evaluation in silico
   Compute melting temperatures and GC % and filter for moderate range sequences
   Iterated genetic algorithm/simulated annealing selects candidate compatible 400 targets
Best 400 targets (800 primers) generated and tested physically for multiplex capabilities at a common melting temperature in common solution.
Of the 400 targets sequenced:
   Filter for sequences that amplified evenly in multiplex
   Moderate read depth window
48 assays designated for MOMA from top performing multiplexed SNPs
   Each SNP has a probe designed in WT/MUT at four mismatch choices (8 probes per assay)
   New nested primers are designed within the 70 bp enriched fragments
   Experimentally amplified at known heterozygous individuals to evaluate amplification efficiency (8×48 TAQMAN in triplicate)

### Apriori Genotyping Informativeness of Each Assay

With known Recipient and Donor genotypes at each assayed SNP, the subset of informative assays is selected.
   Recipient Homozygous sites can be used where the donor is any other genotype
Without donor genotypes, but with clean recipient genotypes available before transplant; donor genotypes can be inferred from plasma data discrepancies.
Genotypes may be learned through sequencing or SNP microarray or application of a MOMA assay on known 0% (clean recipient) samples.

### Post Processing Analysis of Multiplex Assay Performance

Across the experimental cohort the patient specific MOMA probe biases are estimated. Selection iteratively refined such that final donor % call uses only reliable probes. Automatic outlier detection provides patient-specific anomalous genomic regions.

### Reconstruction Experiment

Sensitivity and precision evaluated on reconstructed plasma samples with known mixing ratio.
Evaluated ratios of 1:10, 1:20, 1:100, 1:200, 1:1000.

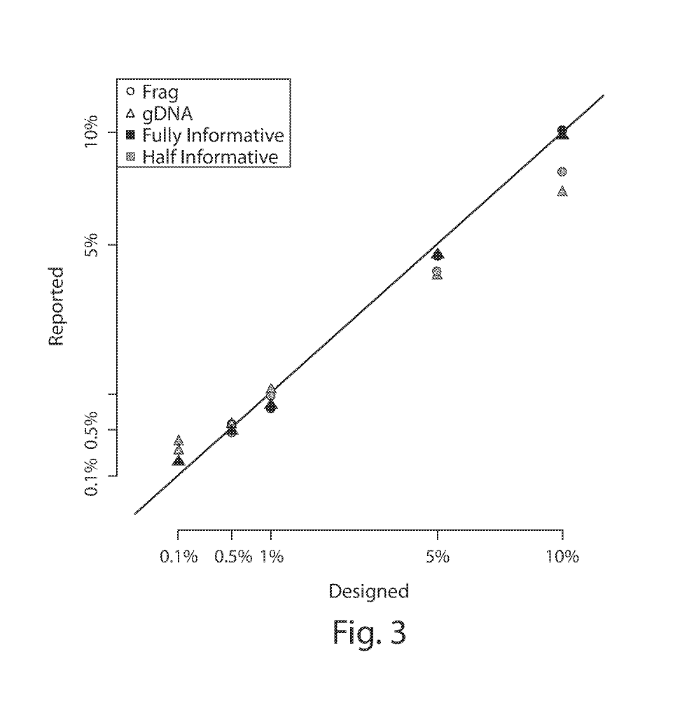

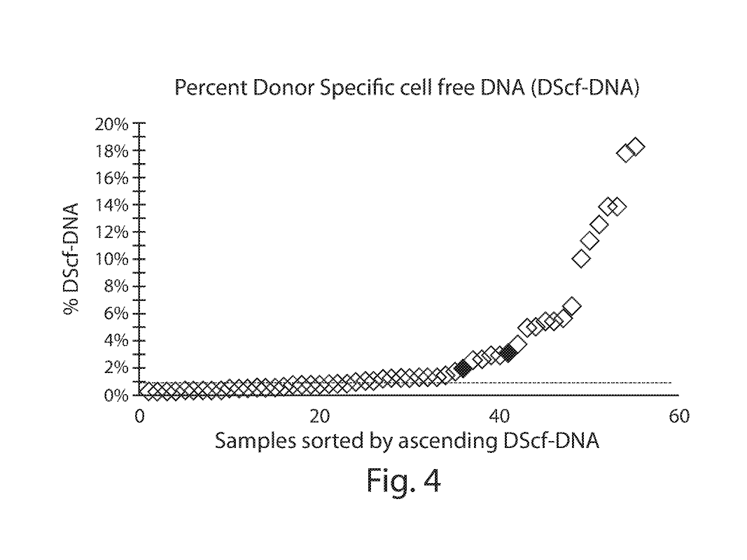

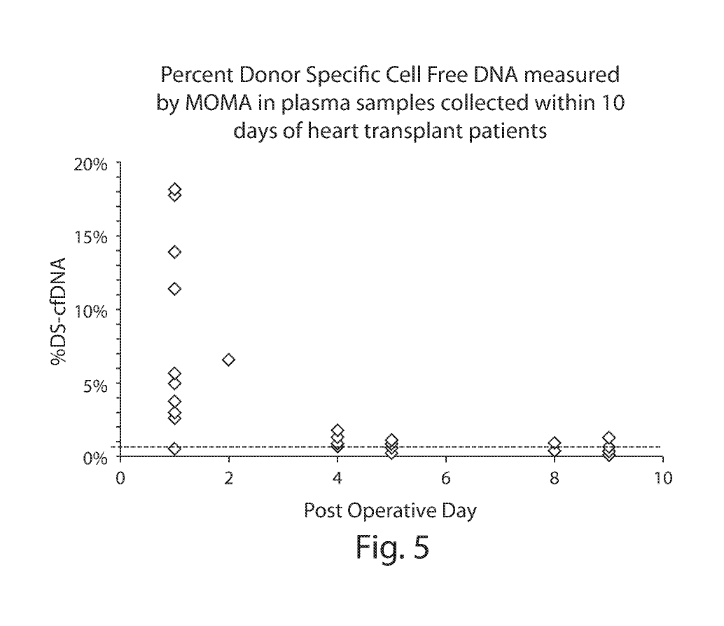

Results of the reconstruction experiment demonstrate proof of concept (FIG. 3). One target is fully informative where there is a homozygous donor against a homozygous recipient (shaded data points). The other target is half informative where there is a heterozygous donor against a homozygous recipient (open data points). In addition, plasma samples from transplant recipient patients were analyzed with a MOMA method (FIG. 4). All data comes from patients who have had biopsies. Dark points denote rejection. Further data shown in FIG. 5, demonstrate that a MOMA method as provided herein worked with real plasma samples. After transplant surgery, the donor percent levels dropped off. Generally, primers for 95 SNV targets as described herein were used.

### Example 2

### With Recipient but not Donor Genotype Information

To work without donor genotype information, the following procedure may be performed to infer informative assays and allow for quantification of donor-specific cell-free DNA in plasma samples. All assays were evaluated for performance in the full information scenario. This procedure thus assumed clean AA/AB/BB genotypes at each assay and unbiased behavior of each quantification. With recipient genotype, assays known to be homozygous in the recipient were selected. Any contamination was attributed to the donor nucleic acids, and the assay collection created a tri-modal distribution with three clusters of assays corresponding to the non-, half, and fully-informative assays. With sufficient numbers of recipient homozygous assays the presence of donor fully informative assays can be assumed.

If recipient genotype is homozygous and known, then if a measurement that is not the recipient genotype is observed, the probes which are truly donor homozygous will have the highest cluster and equal the guess whereas those that are donor heterozygous will be at half the guess. A probability distribution can be plotted and an expectation maximization algorithm (EM) can be employed to infer donor genotype. Such can be used to infer the donor genotype frequency in any one of the methods provided herein. Accordingly, an EM algorithm was used to infer the most likely donor genotypes at all assayed SNV targets. With inferred donor genotypes, quantification may proceed as in the full-information scenario. EM can begin with the assumption that the minor allele ratio found at an assay follows a tri-modal distribution, one for each combination of recipient and donor, given all assays are “AA” in the recipient (or flipped from “BB” without loss of generality). With all donor genotypes unknown, it is possible to bootstrap from the knowledge that any assays exhibiting nearly zero minor allele are donor AA, and the highest is donor BB. Initial guesses for all donor genotypes were recorded, and the mean of each cluster calculated. Enforcing that the donor BB assays' mean is twice that of the donor AB restricts the search. The algorithm then reassigns guessed donor genotypes based on the clusters and built-in assumptions. The process was iterative until no more changes were made. The final result is a set of the most likely donor genotypes given their measured divergence from the background. Generally, every target falls into the model; a result may be tossed if between groups after maximization.

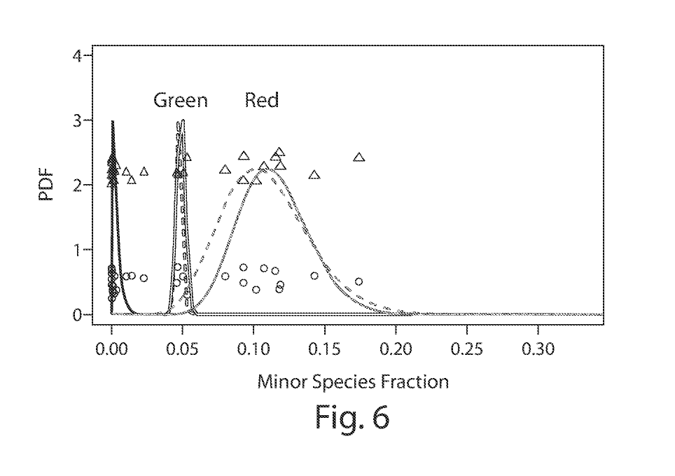

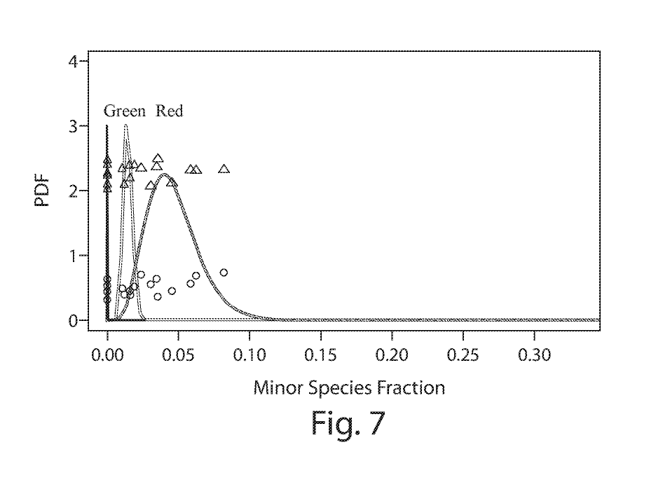

FIG. 6 shows exemplary results from plasma samples handled in this manner. The x-axis is the donor % for any assay found recipient homozygous. The rows of points represent individual PCR assay results. The bottom-most row of circles represents the initial guess of donor genotypes, some AA, some A/B and some BB. Then the solid curves were drawn representing Beta distributions centered on the initial assays, red for homozygous (fully informative) and green for heterozygous (half informative) with black curves representing the distribution of non-informative assays or background noise. The assays were re-assigned updated guesses in the second row. Second row's curves use dashed lines. The top row is the final estimate because no change occurred. Double the peak of the green dashed curve corresponds to the maximum likelihood donor % call, at around 10%, or equal to the mean of the red curve.

A reconstruction experiment (Recon1) using DNA from two individuals were created at 10%, 5%, 1%, 0.5%, and 0.1%. All mixes were amplified with a multiplex library of targets, cleaned, then quantitatively genotyped using a MOMA method. The analysis was performed with genotyping each individual in order to know their true genotypes. Informative targets were determined using prior knowledge of the genotype of the major individual (looking for homozygous sites), and where the second individual was different, and used to calculate fractions (percentage) using informative targets. The fractions were then calculated (depicted in black to denote With Genotype information).

A second reconstruction experiment (Recon2), beginning with two individuals, major and minor were also created at 10%, 5%, 1%, 0.5%, and 0.1%. All mixes were amplified with the multiplex library of targets, cleaned, then quantitatively genotyped using a MOMA method. The analysis was performed with genotyping each individual in order to know their true genotypes. Informative targets were determined using prior knowledge of the genotype of the second individual as described above. The fractions were then calculated (depicted in black to denote With Genotype information).

These reconstructions were run again the next day (Recon3).

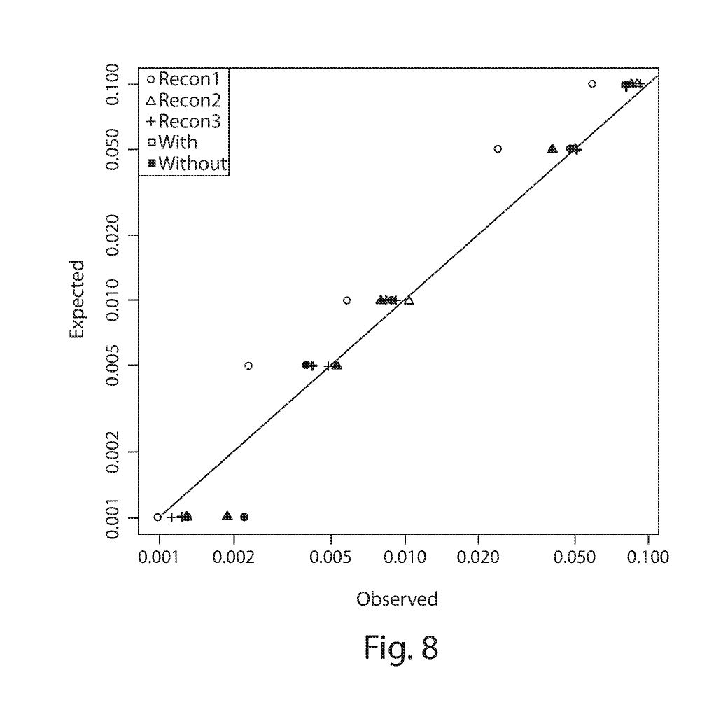

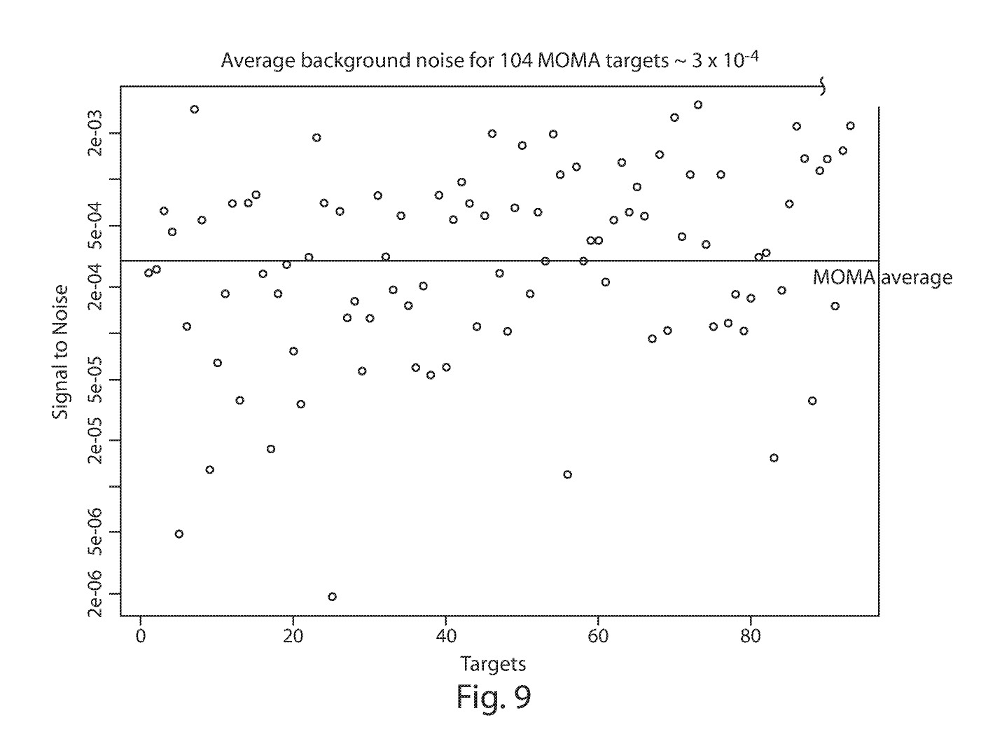

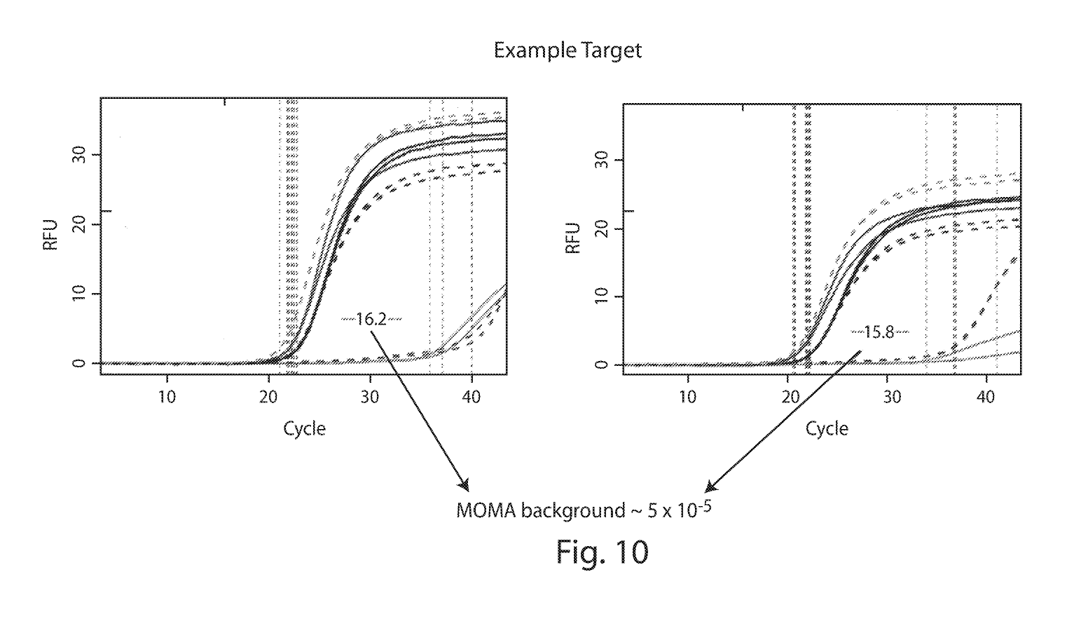

The same reconstruction samples (Recon 1,2,3) were then analyzed again without using genotyping information from the second individual (minor DNA contributor) but only genotyping information available for the first individual (major DNA contributor). Approximately 38-40 targets were used to calculate fractions without genotyping (simulating without donor) shaded (FIG. 8). It was found that each target that was recipient homozygous was possibly useful. The circles were the first guess, just thresholding, those on the right were thought to be fully informative and those on the left not. The triangles along the top were the same targets, but for the final informativity decisions they were recolored. It was found the expectation maximization was superior to simple thresholding.

1. A method of assessing an amount of non-native nucleic acids in a sample from a subject, the sample comprising non-native and native nucleic acids, the method comprising:
for each of a plurality of single nucleotide variant (SNV) targets, performing an amplification-based quantification assay on the sample, or portion thereof, with at least two primer pairs, wherein each primer pair comprises a forward primer and a reverse primer, wherein one of the at least two primer pairs comprises a 3′ penultimate mismatch in a primer relative to one allele of the SNV target but a 3′ double mismatch relative to another allele of the SNV target and specifically amplifies the one allele of the SNV target, and another of the at least two primer pairs specifically amplifies the another allele of the SNV target,
and obtaining or providing results from the amplification-based quantification assays to determine the amount of non-native nucleic acids in the sample.

2. The method of claim 1, wherein the results are provided in a report.

3. The method of claim 1 or 2, wherein the method further comprises determining the amount of the non-native nucleic acids in the sample based on the results.

4. The method of claim 1 or 2, wherein the results comprise the amount of the non-native nucleic acids in the sample.

5. A method of assessing an amount of non-native nucleic acids in a sample from a subject, the sample comprising non-native and native nucleic acids, the method comprising:
obtaining results from an amplification-based quantification assay, for each of a plurality of single nucleotide variant (SNV) targets, performed on the sample, or portion thereof, with at least two primer pairs, wherein each primer pair comprises a forward primer and a reverse primer, wherein one of the at least two primer pairs comprises a 3′ penultimate mismatch in a primer relative to one allele of the SNV target but a 3′ double mismatch relative to another allele of the SNV target and specifically amplifies the one allele of the SNV target, and another of the at least two primer pairs specifically amplifies the another allele of the SNV target, and
assessing the amount of non-native nucleic acids based on the results.

6. The method of claim 5, wherein the amount of the non-native nucleic acids in the sample is based on the results of the amplification-based quantification assays.

7. The method of claim 5 or 6, wherein the results are obtained from a report.

8. The method of any one of the preceding claims, wherein the another primer pair of the at least two primer pairs also comprises a 3′ penultimate mismatch relative to the another allele of the SNV target but a 3′ double mismatch relative to the one allele of the SNV target in a primer and specifically amplifies the another allele of the SNV target.

9. The method of any one of the preceding claims, wherein the amount is the ratio or percentage of non-native nucleic acids to native nucleic acids.

10. The method of any one of the preceding claims, wherein the results are informative results of the amplification-based quantification assays.

11. The method of any one of the preceding claims, wherein the amount is based on informative results of the amplification-based quantification assays.

12. The method of any one of the preceding claims, wherein the method further comprises selecting informative results of the amplification-based quantification assays.

13. The method of claim 12, wherein the selected informative results are averaged.

14. The method of claim 12 or 13, wherein the informative results of the amplification-based quantification assays are selected based on the genotype of the non-native nucleic acids and/or native nucleic acids.

15. The method of any one of the preceding claims, wherein the method further comprises obtaining the genotype of the non-native nucleic acids and/or native nucleic acids.

16. The method of any one of the preceding claims, wherein the method further comprises obtaining the plurality of SNV targets.

17. The method of any one of the preceding claims, wherein the method further comprises obtaining the at least two primer pairs for each of the plurality of SNV targets.

18. The method of any one of the preceding claims, wherein the plurality of SNV targets is at least 90 SNV targets.

19. The method of any one of the preceding claims, wherein the plurality of SNV targets is at least 95 SNV targets.

20. The method of any one of the preceding claims, wherein the plurality of SNV targets is less than 105 SNV targets.

21. The method of any one of the preceding claims, wherein the plurality of SNV targets is less than 100 SNV targets.

22. The method of any one of the preceding claims, wherein the amount of non-native nucleic acids in the sample is at least 0.005%.

23. The method of claim 22, wherein the amount of non-native nucleic acids in the sample is at least 0.01%.

24. The method of claim 23, wherein the amount of non-native nucleic acids in the sample is at least 0.03%.

25. The method of claim 24, wherein the amount of non-native nucleic acids in the sample is at least 0.05%.

26. The method of claim 25, wherein the amount of non-native nucleic acids in the sample is at least 0.1%.

27. The method of claim 26, wherein the amount of non-native nucleic acids in the sample is at least 0.3%.

28. The method of any one of claims 22-27, wherein the amount of non-native nucleic acids in the sample is less than 1.5%.

29. The method of claim 28, wherein the amount of non-native nucleic acids in the sample is less than 1.3%.

30. The method of claim 29, wherein the amount of non-native nucleic acids in the sample is less than 1%.

31. The method of claim 30, wherein the amount of non-native nucleic acids in the sample is less than 0.5%.

32. The method of any one of the preceding claims, wherein when the genotype of the non-native nucleic acids is not known or obtained, the method further comprises:
assessing results based on a prediction of the likely non-native genotype.

33. The method of claim 32, wherein the assessing is performed with an expectation-maximization algorithm.

34. A method of assessing an amount of non-native nucleic acids in a sample from a subject, the sample comprising non-native and native nucleic acids, the method comprising:
obtaining results from 1) a amplification-based quantification assay, for each of a plurality of SNV targets, performed on a sample, or portion thereof, with at least two primer pairs, wherein each primer pair comprises a forward primer and a reverse primer, wherein one of the at least two primer pairs comprises a 3′ penultimate mismatch relative to one allele of the SNV target but a 3′ double mismatch relative to another allele of the SNV target in a primer and specifically amplifies the one allele of the SNV target, and another of the at least two primer pairs specifically amplifies the another allele of the SNV target, and 2) a determination of informative results based on the native genotype and a prediction of the likely non-native genotype, and
providing the results to determine the amount of non-native nucleic acids in the sample.

35. The method of claim 34, wherein the results are provided in a report.

36. The method of claim 34 or 35, wherein the method further comprises determining the amount of non-native nucleic acids in the sample based on the results.

37. The method of any one of claims 34-36, wherein the results comprise the amount of the non-native nucleic acids in the sample.

38. A method of assessing an amount of non-native nucleic acids in a sample from a subject, the sample comprising non-native and native nucleic acids, the method comprising:
obtaining results from 1) a amplification-based quantification assay, for each of a plurality of SNV targets, performed on a sample, or portion thereof, with at least two primer pairs, wherein each primer pair comprises a forward primer and a reverse primer, wherein one of the at least two primer pairs comprises a 3′ penultimate mismatch relative to one allele of the SNV target but a 3′ double mismatch relative to another allele of the SNV target in a primer and specifically amplifies the one allele of the SNV target, and another of the at least two primer pairs specifically amplifies the another allele of the SNV target, and 2) a determination of informative results based on the native genotype and a prediction of the likely non-native genotype, and
assessing the amount of non-native nucleic acids based on the results.

39. The method of claim 38, wherein the amount of the non-native nucleic acids in the sample is based on the results of the amplification-based quantification assays.

40. The method of claim 38 or 39, wherein the results are obtained from a report.

41. The method of any one of claims 34-40, wherein the method further comprises selecting informative results based on the native genotype and prediction of the likely non-native genotype.

42. The method of any one of claims 34-41, wherein expectation-maximization is used to predict the likely non-native genotype.

43. The method of any one of claims 34-42, wherein the another primer pair of the at least two primer pairs also comprises a 3′ penultimate mismatch relative to the another allele of the SNV target but a 3′ double mismatch relative to the one allele of the SNV target in a primer and specifically amplifies the another allele of the SNV target.

44. The method of any one of claims 34-43, wherein the amount is the ratio or percentage of non-native nucleic acids to native nucleic acids.

45. The method of any one of claims 34-44, wherein the method further comprises obtaining the genotype of the native nucleic acids.

46. The method of any one of claims 34-45, wherein the method further comprises obtaining the plurality of SNV targets.

47. The method of any one of claims 34-46, wherein the method further comprises obtaining the at least two primer pairs for each of the plurality of SNV targets.

48. The method of any one of the preceding claims, wherein maximum likelihood is used to calculate the amount of non-native nucleic acids.

49. The method of any one of the preceding claims, wherein the sample comprises cell-free DNA sample and the amount is an amount of non-native cell-free DNA.

50. The method of any one of the preceding claims, wherein the subject is a transplant recipient, and the amount of non-native nucleic acids is an amount of donor-specific cell-free DNA.

51. The method of claim 50, wherein the transplant recipient is a heart transplant recipient.

52. The method of claim 50 or 51, wherein the transplant recipient is a pediatric transplant recipient.

53. The method of any one of the preceding claims, wherein the plurality of amplification-based quantification assays are quantitative PCR assays, such as real time PCR assays or digital PCR assays.

54. The method of any one of the preceding claims, wherein the method further comprises determining a risk in the subject based on the amount of non-native nucleic acids in the sample.

55. The method of claim 54, wherein the risk is a risk associated with a transplant.

56. The method of claim 55, wherein the transplant is a heart transplant.

57. The method of claim 56, wherein the risk associated with a transplant is risk of transplant rejection.

58. The method of any one of claims 54-57, wherein the risk is increased if the amount of non-native nucleic acids is greater than a threshold value.

59. The method of any one of claims 54-57, wherein the risk is decreased if the amount of non-native nucleic acids is less than a threshold value.

60. The method of claim 58 or 59, in the case where the risk is the risk associated with the heart transplant rejection, the threshold value is 1%.

61. The method of claim 58 or 59, in the case where the risk is the risk associated with the heart transplant rejection, the threshold value is 1.3%.

62. The method of any one of the preceding claims, wherein the method further comprises selecting a treatment for the subject based on the amount of non-native nucleic acids.

63. The method of any one of the preceding claims, wherein the method further comprises treating the subject based on the amount of non-native nucleic acids.

64. The method of any one of the preceding claims, wherein the method further comprises providing information about a treatment to the subject based on the amount of non-native nucleic acids.

65. The method of any one of the preceding claims, wherein the method further comprises monitoring or suggesting the monitoring of the amount of non-native nucleic acids in the subject over time.

66. The method of any one of the preceding claims, wherein the method further comprises assessing the amount of non-native nucleic acids in the subject at a subsequent point in time.

67. The method of any one of the preceding claims, wherein the method further comprises evaluating an effect of a treatment administered to the subject based on the amount of non-native nucleic acids.

68. The method of any one of claims 62-67, wherein the treatment is an anti-rejection therapy.

69. The method of any one of the preceding claims, further comprising providing or obtaining the sample or a portion thereof.

70. The method of any one of the preceding claims, further comprising extracting nucleic acids from the sample.

71. The method of any one of the preceding claims, wherein the sample comprises blood, plasma or serum.

72. The method of any one of the preceding claims, wherein the sample is obtained from the subject within 10 days of a heart transplant.

73. A method of determining a plurality of SNV targets, comprising:
a) identifying a plurality of highly heterozygous SNVs in a population of individuals,
b) designing one or more primers spanning each SNV,
c) selecting sufficiently specific primers,
d) computing the melting temperatures and/or GC % of the selected primers and filtering for moderate range sequences,
e) evaluating multiplexing capabilities of primers at a common melting temperature in a common solution, and
f) identifying sequences that are evenly amplified, such as with PCR.

74. The method of claim 73, wherein step a) further comprises selecting SNVs with a Hardy-Weinberg p>0.25 and/or excluding those associated with difficult regions.

75. The method of claim 74, wherein the difficult regions are syndromic regions and/or low complexity regions.

76. The method of any one of claims 73-75, wherein the one or more primers of step b) span a 70 bp window and/or the one or more primers are 16-26 bps in length.

77. The method of any one of claims 73-76, wherein the sufficiently specific primers of step c) are identified with a BLAST analysis.

78. The method of claim 77, wherein the BLAST analysis is against GCRh37.

79. The method of any one of claims 73-78, wherein step d) further includes iterated genetic algorithm and/or simulated annealing.

80. The method of any one of claims 73-79, further comprising obtaining a primer pair for each identified SNV target wherein the primer pair comprises a 3′ penultimate mismatch relative to one allele of the SNV but a 3′ double mismatch relative to another allele of the SNV target in a primer and specifically amplifies the one allele of the SNV target.

81. The method of claim 80, wherein the method further comprises obtaining another primer pair for each identified SNV, wherein the another primer pair specifically amplifies the another allele of the SNV target.

82. The method of claim 81, wherein the another primer pair comprises a 3′ penultimate mismatch relative to the another allele of the SNV but a 3′ double mismatch relative to the one allele of the SNV in a primer.

83. The method of any one claims 73-82, wherein the plurality of SNV targets identified is at least 90 SNV targets.

84. The method of claim 83, wherein the plurality of SNV targets is at least 95 SNV targets.

85. The method of any one of claims 73-84, wherein the plurality of SNV targets identified is less than 105 SNV targets.

86. The method of claim 85, wherein the plurality of SNV targets is less than 100 SNV targets.

87. A composition or kit comprising,
a primer pair, for each of a plurality of SNV targets, wherein each primer pair comprises a 3′ penultimate mismatch relative to one allele of a SNV target but a 3′ double mismatch relative to another allele of the SNV target in a primer and specifically amplifies the one allele of the SNV target.

88. The composition or kit of claim 87, further comprising another primer pair for each of the plurality of SNV targets wherein the another primer pair specifically amplifies the another allele of the SNV target.

89. The composition or kit of claim 87 or 88, wherein the plurality of SNV targets is at least 90 SNV targets.

90. The composition or kit of claim 89, wherein the plurality of SNV targets is at least 95 SNV targets.

91. The composition or kit of any one of claims 87-90, wherein the plurality of SNV targets is less than 105 SNV targets.

92. The composition or kit of claim 91, wherein the plurality of SNV targets is less than 100 SNV targets.

93. The composition or kit of any one of claims 87-92, further comprising a buffer.

94. The composition or kit of any one of claims 87-93, further comprising a polymerase.

95. The composition or kit of any one of claims 87-94, further comprising a probe.

96. The composition or kit of claim 95, wherein the probe is a fluorescent probe.

97. The composition or kit of any one of claims 87-96, further comprising instructions for use.

98. The composition or kit of claim 97, wherein the instructions for use are instructions for determining the amount of non-native nucleic acids in a sample.

99. The composition or kit of claim 98, wherein the sample is from a heart transplant recipient.

100. The composition or kit of claim 99, wherein the sample is from a pediatric heart transplant recipient.

101. A method of inferring non-native nucleic acid genotype:
obtaining informative non-native nucleic levels for each of a plurality of single nucleotide variant (SNV) targets,
assigning the levels to one of two distributions, one of which is for fully informative levels and the other is for half informative levels, with a maximum likelihood or expectation maximization step.

102. The method of claim 101, wherein the informative non-native nucleic acid levels are obtained by removing levels that are determined to be of native nucleic acids.

103. The method of claim 101 or 102, wherein the method further comprises removing levels that represent a no call or erroneous call.

104. The method of any one of claims 101-103, wherein the levels are determined with sequencing, such as with next generation sequencing.

105. The method of any one of claims 101-104, wherein the levels are obtained from a amplification-based quantification assay performed for each of the plurality of SNV targets.

106. The method of claim 105, wherein the amplification-based quantification assay is performed with at least two primer pairs for each of the plurality of SNV targets, wherein each primer pair comprises a forward primer and a reverse primer, wherein one of the at least two primer pairs comprises a 3′ penultimate mismatch relative to one allele of the SNV target but a 3′ double mismatch relative to another allele of the SNV target in a primer and specifically amplifies the one allele of the SNV target, and another of the at least two primer pairs specifically amplifies the another allele of the SNV target.

107. The method of claim 106, wherein the another primer pair of the at least two primer pairs also comprises a 3′ penultimate mismatch relative to the another allele of the SNV target but a 3′ double mismatch relative to the one allele of the SNV target in a primer and specifically amplifies the another allele of the SNV target.

108. The method of any one of claims 101-107, wherein the method further comprises providing the assigned levels.

109. The method of any one of claims 101-108, wherein the method further comprises obtaining the amount of non-native nucleic acids based on the assignment of the levels.

110. The method of any one of claims 101-109, wherein the method further comprises providing the amount of non-native nucleic acids based on the assignment of the levels.

111. A method comprising:
obtaining the levels assigned as fully informative or half informative or amount of non-nucleic acids based on the assignment according to a method of any one of claims 101-110, and
assessing a risk in a subject based on the levels or amount.

112. The method of claim 111, wherein the subject is a recipient of a transplant.

113. The method of claim 111 or 112, wherein a treatment or information about a treatment is given to the subject based on the assessed risk.

114. The method of claim 113, wherein the treatment is an anti-rejection therapy.

115. The method of any one of claims 111-113, wherein the method further comprises monitoring or suggesting the monitoring of the amount of non-native nucleic acids in the subject over time.
